(19) United States
(12) Patent Application Publication
(10) Pub. No.: US 2013/0189367 A1
(43) Pub. Date: Jul. 25, 2013

(54) NANOVECTORS FOR TARGETED GENE SILENCING AND CYTOTOXIC EFFECT IN CANCER CELLS

(71) Applicants: Miqin Zhang, Bothell, WA (US); Hyejung Mok, Seoul (KR)

(72) Inventors: Miqin Zhang, Bothell, WA (US); Hyejung Mok, Seoul (KR)

(73) Assignee: University of Washington through its Center for Commercialization, Seattle, WA (US)

(21) Appl. No.: 13/562,188

(22) Filed: Jul. 30, 2012

Related U.S. Application Data
(60) Provisional application No. 61/513,169, filed on Jul. 29, 2011.

(51) Int. Cl. A61K 9/16

(52) U.S. Cl. CPC A61K 9/1676
USPC 424/493; 514/44 R; 435/34; 435/375; 514/44 A

(57) ABSTRACT

Nanoparticle having a coating comprising a polyethylenimine and a chitosan-polyethylene oxide oligomer copolymer, and methods for making and using the nanoparticle. The nanoparticle can have a core that includes a material that imparts magnetic resonance imaging activity to the particle and, optionally, include one or more of an associated therapeutic agent, targeting agent, and fluorescent agent.

### CROSS-REFERENCE TO RELATED APPLICATION

This application claims the benefit of U.S. Patent Application No. 61/513,169, filed Jul. 29, 2011, expressly incorporated herein by reference in its entirety.

### STATEMENT OF GOVERNMENT LICENSE RIGHTS

This invention was made with government support under Grant Nos. RO1CA134213 and RO1EB006043 awarded by the National Institutes of Health. The government has certain rights in the invention.

### STATEMENT REGARDING SEQUENCE LISTING

The sequence listing associated with this application is provided in text format in lieu of a paper copy and is hereby incorporated by reference into the specification. The name of the text file containing the sequence listing is 39712_SEQ_FINAL.txt. The text file is 1 KB; was created on Jul. 30, 2012; and is being submitted via EFS-Web with the filing of the specification.

### BACKGROUND OF THE INVENTION

RNA interference (RNAi) is a mechanism by which living cells regulate gene expression using small RNA molecules. Short interfering RNA (siRNA) is double stranded, non-coding RNA, which binds to complementary mRNA to direct gene silencing through argonaute, an endonuclease within the RNA-induced silencing complex (RISC). RNAi is a rapidly developing frontier in the field of gene therapy that shows tremendous potential as a therapeutic approach for the treatment of various diseases caused by aberrant gene expression, and cancer is a prime target where this technology can be applied. By silencing the genes that contribute to uncontrolled cell growth, siRNA treatments can curb pathogenesis and potentially induce cancer cell death. However, unlike conventional chemotherapeutics, RNA molecules are anionic, hydrophilic, and cannot be internalized by cells through passive diffusion. Furthermore, upon internalization they are ineffectively trafficked by cancer cells, hindering their potency. Therefore, there is considerable interest in the development of safe and effective carriers to facilitate both the delivery and intracellular trafficking of siRNA.

A broad spectrum of siRNA delivery constructs, including viral vectors, liposomes, cationic polymers and dendrimers, cell-penetrating peptides, semiconductor quantum dots, and gold and magnetic nanoparticles have been investigated. Non-viral methods are preferred over viral approaches because they present a lower risk of immunogenicity, and do not produce oncologic side effects. Thus, in recent years a great deal of attention has been paid towards the development of non-viral carriers for siRNA delivery. A common characteristic among these constructs is their net positive charge, which contributes to both complex formation with the anionic siRNA, and interaction with the negatively charged cell membrane. From these carriers, several formulations (e.g., liposomes and cationic polymers) have emerged and shown great potential for clinical implementation for cancer therapy. For example, a lipoplex formulation is currently under Phase I clinical investigation for the treatment of liver cancer. A liposome formulation for the treatment of solid tumors has also been developed.

Although significant advances have been made in developing gene delivery vehicles, poor site specificity, low efficacy in gene silencing, and lack of non-invasive delivery monitoring remain major hurdles towards clinical advancement.

### SUMMARY OF THE INVENTION

In one aspect, the present invention provides a nanoparticle having a coating comprising a polyethylenimine and a chitosan-polyethylene oxide oligomer copolymer. In certain embodiments, the nanoparticle has a core that includes a material that imparts magnetic resonance imaging activity to the particle. The nanoparticle can further include one or more of a therapeutic agent that can be delivered by the particle, a targeting agent to target the nanoparticle to a site of interest, and a fluorescent agent that allows for fluorescence imaging of the particle.

In one embodiment, the nanoparticle, comprises:

(a) a core having a surface and comprising a core material; and

(b) a coating on the surface of the core, the coating comprising
(i) a copolymer comprising a chitosan and a poly(ethylene oxide) oligomer; and
(ii) a polyethylenimine having an average molecular weight from about 500 to about 10,000 g/mole, or
(iii) a polyethylenimine having primary, secondary, and tertiary amine groups, wherein at least a portion of primary amine groups are modified to provide amide groups.

In certain embodiments, the nanoparticle further comprises a therapeutic agent covalently coupled to the coating. In other embodiments, the nanoparticle further comprises a targeting agent covalently coupled to the coating. In further embodiments, the nanoparticle further comprises a therapeutic agent covalently coupled to the coating and a targeting agent covalently coupled to the coating.

In one embodiment, the polyethylenimine having an average molecular weight from about 500 to about 10,000 g/mole has an average molecular weight from about 500 to about 2,000 g/mole.

In one embodiment, the polyethylenimine having at least a portion of primary amine groups modified to provide amide groups has an average molecular weight from about 600 to about 60,000 g/mole. In certain embodiments, the portion of primary amine groups modified to provide amide groups introduce carboxylate groups to the polyethylenimine. In certain embodiments, the polyethylenimine modified to provide amide groups is reactive under acidic conditions to reverse the modification and regenerate the primary amine groups.

Suitable therapeutic agents include small organic molecules, peptides, aptamers, proteins, and nucleic acids. In certain embodiments, the therapeutic agent is an RNA (e.g., siRNA) or a DNA. In certain embodiments, the therapeutic agent is covalently coupled to the coating is coupled through a cleavable linkage.

Suitable targeting agents include small organic molecules, peptides, aptamers, proteins, and nucleic acids. In certain embodiments, the targeting agent is a chlorotoxin, RGD, or VHPNKK.

In certain embodiments, the nanoparticles of the invention further comprise a fluorescent agent.

In certain embodiments, the nanoparticle's core material is a magnetic material.

In certain embodiments, the nanoparticle's coating includes a copolymer that is a graft copolymer having a chitosan backbone and poly(ethylene oxide) oligomer side chains.

In another aspect, the invention provides a composition comprising a nanoparticle of the invention and a carrier suitable for administration to a warm-blooded subject.

In a further aspect of the invention, a method for detecting cells or tissues by magnetic resonance imaging is provided. In one embodiment, the method includes:

(a) contacting cells or tissues of interest with a nanoparticle of the invention; and

(b) measuring the level of binding of the nanoparticle, wherein an elevated level of binding, relative to normal cells or tissues, is indicative of binding to the cells or tissues of interest.

In another aspect, the invention provides a method for treating a tissue. In on embodiment, the method includes contacting a tissue of interest with a nanoparticle of the invention.

In a further aspect of the invention, a method for silencing or reducing the expression level of a gene is provided. In one embodiment, the method includes contacting a cell of interest with a nanoparticle of the invention.

### DESCRIPTION OF THE DRAWINGS

The foregoing aspects and many of the attendant advantages of this invention will become more readily appreciated as the same become better understood by reference to the following detailed description, when taken in conjunction with the accompanying drawings.

FIG. 1A is an illustration of a representative cancer targeting magnetic siRNA nanovector (NP-siRNA-CTX) of the invention.

FIG. 1B is a schematic illustration of the preparation of a representative cancer targeting magnetic siRNA nanovector (NP-siRNA-CTX) of the invention.

FIGS. 2A-2D illustrate the assessment and optimization of siRNA attachment to NP and the subsequent verification of linkage chemistry. FIG. 2A illustrates the results of a gel retardation assay analyzing siRNA attachment onto base NPs at different weight ratios of NP to siRNA (the arrow points to the band of interest). FIG. 2B is the corresponding quantitative analysis (gel shown in FIG. 2A) of degree of siRNA binding. FIG. 2C illustrates the results of a gel retardation assay analyzing siRNA release profiles of NP-siRNA complex through treatment with heparin (electrostatic disrupting agent) and glutathione (reducing agent) (the arrow points to the band of interest). FIG. 2D is the corresponding quantitative analysis (gel shown in c) of percent siRNA released from NPs.

FIGS. 3A-3C illustrate the colloidal-stability of NP-siRNA in various media assessed at 37° C. over a period of 24 hours. FIG. 3A illustrates the hydrodynamic size of NP-siRNA in solutions of various NaCl concentrations and corresponding photo images of the tested samples. FIG. 3B illustrates the hydrodynamic size of NP-siRNA in solutions of varying pH and corresponding photo images of the tested samples. FIG. 3C illustrates the hydrodynamic size of NP-siRNA in DMEM/10% FBS cell culture medium measured over time.

FIGS. 4A-4C illustrate receptor-mediated internalization of siRNA by target cells monitored by flow cytometry and MRI. FIG. 4A illustrates flow cytometry analysis of siRNA internalization by C6/GFP+ cells 2 hrs post treatment with either NP-siRNA or NP-siRNA-CTX; also shown is the result for cells receiving no nanovector treatment (Untreated) as a reference. The diagram above the bar charts conceptually illustrates the interaction of the non-targeting NP-siRNA and targeting NP-siRNA-CTX with cells. FIG. 4B illustrates R₂ relaxivity maps of C6/GFP+ cells treated with either NP-siRNA or NP-siRNA-CTX acquired 2 hrs post treatment; also shown are the images for untreated cells and agarose as references. FIG. 4C is the corresponding R₂ values for each condition shown in FIG. 4B.

FIG. 5 illustrates confocal fluorescence images of cells for assessment of endosomal escape. C6 cells were treated with either calcein alone (negative control, first row) or co-treated with calcein+NP-siRNA-CTX (test sample, second row), or calcein+ PEI/siRNA (positive control, third row). The DIC images are provided to co-localize the cells in fluorescence images. The scale bar represents 20 μm.

FIGS. 6A-6D illustrate receptor-mediated GFP knockdown characterized at the protein level by flow cytometry. FIG. 6A shows results for untreated C6GFP+ cells. FIG. 6B shows results for cells treated with NP-CTX (20 μg/ml of Fe; iron equivalence to siRNA containing samples). FIG. 6C shows results for cells treated with NP-siRNA (2 μg/ml siRNA). FIG. 6D shows results for cells treated with NP-siRNA-CTX (2 μg/ml siRNA).

FIGS. 7A and 7B illustrate receptor-mediated GFP knockdowns analyzed at the RNA level by qRT-PCR and cell viability analyzed using Alamar blue, respectively. FIG. 7A illustrates GFP expression of C6/GFP+ analyzed as untreated or 48 hrs post-treatment with one of the following reagents: siRNA (2 μg/ml), Lipofectamine 2000, Dharmafect 4, PEI/siRNA, NP-siRNA, and NP-siRNA-CTX (all at 2 μg/ml of siRNA). As a reference, cells not expressing GFP (i.e. GFP−) were also analyzed (GFP− Control). Expression is normalized to B-actin levels. FIG. 7B illustrates viability of C6/GFP+ glioma cells 48 hrs post-treatment with one of above transfection reagents, determined by percent reduction of Alamar Blue and normalized to untreated cells.

FIG. 8 illustrates confocal fluorescence images of C6GFP+ cells illustrating targeted siRNA delivery and enhanced knockdown of gene expression. Cells were imaged 48 hrs post-treatment with NP-siRNA (second row) or NP-siRNA-CTX (third row), with untreated cells (first row) as a reference. The scale bar corresponds to 20 μm.

FIGS. 9A and 9B illustrate a representative nanovector of the invention and methods for its preparation. FIG. 9A illustrates the preparation of the amine blocked PEI. FIG. 9B illustrates a representative multifunctional NP-PEIb-siRNA-CTX nanovector and its intracellular uptake, extracellular trafficking, and processing in tumor cells.

FIGS. 10A-10D illustrate the characterization of the primary amine-blocked and pyridyldithiol-activated PEI. FIG. 10A illustrates the relative amount of primary amine groups after amine blocking reaction by citraconic anhydride and pyridyldithiol reaction by SPDP using two types of PEI (PEIa and PEIb), respectively (PEIa, less amine blocked PEI; and PEIb, highly amine blocked PEI). FIG. 10B illustrates the cytotoxicities of PEIa and PEIb in C6 rat glioma cells, where the naked PEI is presented as a control. FIG. 10C illustrates the relative amount of exposed primary amine groups in PEIb after incubation for 24 hrs at various pH conditions. FIG. 10D illustrates the cytotoxicities on C6 cells by primary amine recovered PEIb at pH 0.3, where the naked PEI is presented as a reference.

FIGS. 11A and 11B illustrate gel electrophoresis to confirm the amount of siRNA on NP and covalent conjugation of siRNA to nanovector. FIG. 11A illustrates agarose gel electrophoresis of NP-PEIb-siRNA-CTX after conjugation of different amounts of siRNA on the nanovector, where the naked siRNA serves as a control. FIG. 11B illustrates an gel retardation assay for siRNA physically mixed with naked PEI or NP-PEIb at a PEI/siRNA weight ratio of 0.18.

FIGS. 12A-12D compare physical properties of various iron oxide nanoparticles. FIG. 12A illustrates surface charge. FIG. 12B compares hydrodynamic size of different nanoparticles at pH 7.4 and pH 6.4. FIG. 12C illustrates the stability of NP-PEIb-siRNA-CTX in PBS, assessed in terms of hydrodynamic size profile. FIG. 12D is an TEM image of NP-PEIb-siRNA-CTX (scale bar=20 nm).

FIGS. 13A-13D illustrate magnetic properties of NP-PEIb-siRNA-CTX and cellular uptake of various nanoparticles. FIG. 13A compares R2 relaxation as a function of Fe concentration for NP-PEIb-siRNA-CTX. FIG. 13B compares R2 maps of gel phantoms containing NP-PEIb-siRNA-CTX at different Fe concentrations. FIG. 13C compares intracellular uptake of different nanoparticles by C6 cells in terms of Fe content determined by the colorimetric ferrozine-based assay. All nanoparticles were treated with cells at pH 6.4 at a Fe concentration of 4 μg/mL. FIG. 13D compares R2 maps of gel phantoms containing cells treated with different nanoparticles. All cells were treated with nanoparticles at a concentration of 20 μg/mL.

FIGS. 14A and 14B compare cytotoxic and gene silencing effects of various nanoparticles in C6 cells at pH 7.4 and pH 6.2. FIG. 14A compares relative cell viability after cells were incubated with four different types of NPs for 48 hrs at a Fe concentration of 4 μg/well in serum-containing medium. FIG. 14B compares relative GFP expression by cells treated with NP-PEIb-siRNA-CTX at various Fe concentrations, showing gene silencing effect. *p=0.0001, **p<0.002.

### DETAILED DESCRIPTION OF THE INVENTION

The present invention provides a nanoparticle having a coating comprising a polyethylenimine and a chitosan-polyethylene oxide oligomer copolymer. In certain embodiments, the nanoparticle has a core that includes a material that imparts magnetic resonance imaging activity to the particle. The nanoparticle can further include one or more of a therapeutic agent that can be delivered by the particle, a targeting agent to target the nanoparticle to a site of interest, and a fluorescent agent that allows for fluorescence imaging of the particle. The therapeutic, targeting, and fluorescent agents can be coupled to the particle's copolymer coating. Methods for making and using the nanoparticles are also provided.

In one aspect, the invention provides a multifunctional nanoparticle that includes polyethylenimine (PEI) as a component. Polyethylenimine is a well-characterized and commercialized cationic polymer having a high charge density (a nitrogen capable of protonation per every third atom). Polyethylenimine has been used as a coating material of inorganic nanoparticles as well as itself being a carrier for gene delivery due to its strong electrostatic affinity to nucleotides and efficient endosomal escape via the proton sponge effect after intracellular uptake. However, its non-specific cytotoxicity caused by destabilization of cellular and mitochondrial membranes and activation of intracellular apoptotic signals limit its clinical application.

The disadvantages of using polyethylenimine for therapeutic agent delivery have been overcome by the present invention. In the present invention, polyethylenimine is included as a component of the nanoparticle (or nanovector) coating. In one embodiment, a relatively low molecular weight polyethylenimine is used. In another embodiment, a polyethylenimine having at least a portion of its primary amine groups modified to provide amide groups is used. In each embodiment, the resulting nanoparticle does not suffer from the undesired disadvantages associated with “native” polyethylenimine noted above.

In one aspect, the invention provides a polyethylenimine-containing nanoparticle. In one embodiment, the nanoparticle, comprises:
(a) a core having a surface and comprising a core material; and
(b) a coating on the surface of the core, the coating comprising
   (i) a copolymer comprising a chitosan and a poly(ethylene oxide) oligomer; and
   (ii) a polyethylenimine having an average molecular weight from about 500 to about 10,000 g/mole, or
   (iii) a polyethylenimine having primary, secondary, and tertiary amine groups, wherein at least a portion of primary amine groups are modified to provide amide groups.

In certain embodiments, the polyethylenimine is present in an amount from about 0.2 to about 0.7 percent by weight based on the total weight of the nanoparticle.

Suitable polyethylenimines include linear and branched polyethylenimines.

For the nanoparticle containing a low molecular weight polyethylenimine, the polyethylenimine having an average molecular weight from about 500 to about 10,000 g/mole has an average molecular weight from about 500 to about 5,000 g/mole. In certain embodiments, the polyethylenimine has an average molecular weight from about 500 to about 2,000 g/mole. In other embodiments, the polyethylenimine has an average molecular weight from about 1,000 to about 2,500 g/mole. In further embodiments, the polyethylenimine has an average molecular weight from about 1,000 to about 1,500 g/mole.

For the nanoparticle containing a polyethylenimine having at least a portion of primary amine groups modified to provide amide groups, the polyethylenimine has an average molecular weight from about 600 to about 60,000 g/mole. In certain embodiments, the polyethylenimine has an average molecular weight from about 5,000 to about 60,000 g/mole. In other embodiments, the polyethylenimine has an average molecular weight from about 10,000 to about 50,000 g/mole. In further embodiments, the polyethylenimine has an average molecular weight from about 20,000 to about 60,00 g/mole.

In certain embodiments, from about 15 to about 65 percent of the polyethylenimine primary amine groups are modified to provide amide groups. Depending on the nature of the reagent used for modifying the polyethylenimine (e.g., capping the primary amino groups), the resulting amide-containing polyethylenimine can further include carboxylate groups (e.g., anhydride or dicarboxylic acid reagents) or sulfonate groups (e.g., sulfonic acid reagents). In these embodiments, the positive charge associated with the primary amine groups is converted to a negative charge. In certain embodiments, the polyethylenimine modified to include amide groups is reactive under acidic conditions to reverse the modification and regenerate the primary amine groups. In this way, the polyethylenimine's cytotoxic property is advantageously regenerated at the site of action (e.g., acidic pH endosome of tumor cell).

In certain embodiments, the polyethylenimine is reacted with a maleic anhydride or derivative thereof (e.g., citraconic anhydride) and the amidation imparts a carboxylate to the polyethylenimine. Other suitable amidation reagents include polyhistidine, beta-amino acids, and reagents that produce sulfonamides. In one embodiment, the amide is a sulfonamide.

In addition to the polyethylenimine, the nanoparticle coating includes a copolymer comprising a chitosan and a poly(ethylene oxide) oligomer. In one embodiment, the copolymer is a graft copolymer having a chitosan backbone and pendant poly(ethylene oxide) oligomer side chains.

Chitosan is a linear polysaccharide composed of randomly distributed β-(1-4)-linked D-glucosamine (deacetylated unit) and N-acetyl-D-glucosamine (acetylated unit). Suitable chitosans useful in making the copolymers useful in the invention have a molecular weight (weight average, Mw) of from about 0.3 to about 50 kDa. In certain embodiments, the chitosan has a molecular weight of from about 0.5 to about 15 kDa. In one embodiment, the chitosan has a molecular weight of about 10 kDa. Suitable chitosans include oxidatively degraded chitosans prepared from commercially available chitosan.

The copolymer also includes a plurality of poly(ethylene oxide) oligomers. In one embodiment, poly(ethylene oxide) oligomers are grafted to the chitosan's backbone to provide a copolymer having pendant poly(ethylene oxide) oligomer side chains.

Suitable poly(ethylene oxide) oligomers include poly(ethylene oxides) (PEO or PEG) and poly(ethylene oxide) copolymers such as block copolymers that include poly(ethylene oxide) and poly(propylene oxide) (e.g., PEO-PPO and PEO-PPO-PEO). In one embodiment, the poly(ethylene oxide) oligomer is a poly(ethylene oxide). In certain embodiments, poly(ethylene oxide) oligomer has a molecular weight (weight average, Mw) of from about 0.3 to about 40 kDa. In others embodiments, the poly(ethylene oxide) oligomer has a molecular weight of from about 1.0 to about 10 kDa. In certain embodiments, the poly(ethylene oxide) oligomer has a molecular weight of about 2 kDa.

Representative chitosan-poly(ethylene oxide) oligomer copolymers include from about 2 to about 50 weight percent poly(ethylene oxide) oligomer. In one embodiment, the copolymer includes from about 5 to about 25 weight percent poly(ethylene oxide) oligomer.

Representative chitosan-poly(ethylene oxide) oligomer graft copolymers have a degree of poly(ethylene oxide) oligomer substitution of from about 0.01 to about 0.5. In certain embodiments, the graft copolymers have a degree of poly(ethylene oxide) oligomer substitution from about 0.01 to about 0.2. As used herein, the term “degree of substitution” or “DS” refers to the fraction of glucosamine repeating units in the chitosan that are substituted with a poly(ethylene oxide) oligomer. For DS=1.0, 100% of the glucosamine units are substituted with the poly(ethylene oxide) oligomer.

The copolymer forms a coating on the core surface. The copolymer is anchored to the core surface (e.g., oxide surface) by interactions between the core surface and the amine and hydroxyl groups on the copolymer's chitosan backbone. It is believed that the coating is a multi-layered mesh that encapsulates the core. The polyethylenimine is associated to the copolymer.

The nanoparticle includes a core material. For magnetic resonance imaging applications, the core material is a material having magnetic resonance imaging activity (e.g., the material is paramagnetic). In certain embodiments, the core material is a magnetic material. In other embodiments, the core material is a semiconductor material. Representative core materials include ferrous oxide, ferric oxide, silicon oxide, polycrystalline silicon oxide, silicon nitride, aluminum oxide, germanium oxide, zinc selenide, tin dioxide, titanium, titanium dioxide, nickel titanium, indium tin oxide, gadolinium oxide, stainless steel, gold, and mixtures thereof.

The particle of the invention has nanoscale dimensions. Suitable particles have a physical size less than about 50 nm. In certain embodiments, the nanoparticles have a physical size from about 10 to about 50 nm. In other embodiments, the nanoparticles have a physical size from about 10 to about 30 nm. As used herein, the term “physical size” refers the overall diameter of the nanoparticle, including core (as determined by TEM) and coating thickness. Suitable particles have a mean core size of from about 2 to about 25 nm. In certain embodiments, the nanoparticles have a mean core size of about 7 nm. As used herein, the term “mean core size” refers to the core size determined by TEM. Suitable particles have a hydrodynamic size less than about 250 nm. In certain embodiments, the nanoparticles have a hydrodynamic size from about 20 to about 250 nm. In certain embodiments, the nanoparticles have a hydrodynamic size of about 33 nm. As used herein, the term “hydrodynamic size” refers the radius of a hard sphere that diffuses at the same rate as the particle under examination as measured by DLS. The hydrodynamic radius is calculated using the particle diffusion coefficient and the Stokes-Einstein equation given below, where k is the Boltzmann constant, T is the temperature, and η is the dispersant viscosity:

$${R}_{H} = \frac{kT}{6 π η D} .$$

A single exponential or Cumulant fit of the correlation curve is the fitting procedure recommended by the International Standards Organization (ISO). The hydrodynamic size extracted using this method is an intensity weighted average called the Z average.

The nanoparticles of the invention include the copolymer coated nanoparticles described above that further include one or more other agents. Thus, in other embodiments, the nanoparticles of the invention further include one or more of a therapeutic agent that can be delivered by the particle, a targeting agent to target the nanoparticle to a site of interest, or a fluorescent agent that allows for fluorescence imaging of the particle. The therapeutic, targeting, and fluorescent agents can be coupled to the particle's copolymer coating.

Therapeutic Agents.

Therapeutic agents effectively delivered by the nanoparticles of the invention include small organic molecules, peptides, aptamers, proteins, and nucleic acids. In certain embodiments, the therapeutic agent is an RNA or a DNA (e.g., an siRNA).

Suitable therapeutic agents include conventional therapeutic agents, such as small molecules; biotherapeutic agents, such as peptides, proteins, and nucleic acids (e.g., DNA, RNA, cDNA, siRNA); and cytotoxic agents, such as alkylating agents, purine antagonists, pyrimidine antagonists, plant alkaloids, intercalating antibiotics, antitumor antibiotics (e.g., trastuzumab), binding epidermal growth factor receptors (tyrosine-kinase inhibitors), aromatase inhibitors, anti-metabolites (e.g., folic acid analogs, methotrexate, 5-fluoruracil), mitotic inhibitors (e.g., taxol, paclitaxel, docetaxel), growth factor inhibitors, cell cycle inhibitors, enzymes, topoisomerase inhibitors, biological response modifiers, anti-hormones, anti-androgens, and various cytokines for immunotherapy. Representative cytotoxic agents include BCNU, cisplatin, gemcitabine, hydroxyurea, paclitaxel, temozomide, topotecan, fluorouracil, vincristine, vinblastine, procarbazine, dacarbazine, altretamine, cisplatin, methotrexate, mercaptopurine, thioguanine, fludarabine phosphate, cladribine, pentostatin, fluorouracil, cytarabine, azacitidine, vinblastine, vincristine, etoposide, teniposide, irinotecan, docetaxel, doxorubicin, daunorubicin, dactinomycin, idarubicin, plicamycin, mitomycin, bleomycin, tamoxifen, flutamide, leuprolide, goserelin, aminoglutethimide, anastrozole, amsacrine, asparaginase, mitoxantrone, mitotane, and amifostine.

Suitable therapeutic drugs include siRNAs and antitumor tumor drugs than function in cytoplasma.

In one embodiment, the invention provides a nanoparticle, comprising:

(a) a core comprising a magnetic material and having a surface; and

(b) a coating on the surface of the core, the coating comprising a polyethylenimine and a copolymer comprising a chitosan and a poly(ethylene oxide) oligomer;

(c) a targeting agent covalently coupled to the copolymer; and

(d) a therapeutic agent.

In the above embodiment, the therapeutic agent can be covalently coupled to the copolymer or non-covalently (e.g., ionic) associated with the copolymer. For therapeutic agent delivery, the therapeutic agent can be covalently linked (e.g., through a cleavable linkage), physically adsorbed to (e.g., electrostatic or van der Waals interactions), or embedded within the nanoparticle's copolymer coating.

In certain embodiments, the therapeutic agent is covalently coupled to the coating through a cleavable linkage. Suitable cleavable linkages include linkages cleavable under acidic conditions such as in the acidic microenvironment of cancer cell (e.g., pH less than physiological pH, from about 4.0 to about 6.8. Representative cleavable linkages include acetal, hydrazone, orthoester, and thioester linkages.

For embodiments of the nanoparticle that include a nucleic acid therapeutic agent (e.g., siRNA), the nanoparticle is a nanovector.

Targeting Agents.

Suitable targeting agents include compounds and molecules that direct the nanoparticle to the site of interest. Suitable targeting agents include tumor targeting agents. Representative targeting agents include small molecules, peptides, proteins, aptamers, and nucleic acids. Representative small molecule targeting agents include folic acid, methotrexate, non-peptidic RGD mimetics, vitamins, and hormones. Representative peptide targeting agents include RGD (avβ3 integrin), chlotoxin (MMP2), and VHPNKK (endothlial vascular adhesion molecules). Representative protein targeting agents include antibodies against the surface receptors of tumor cells, such as monoclonal antibody A7 (colorectal carcinoma), herceptin (Her2/ner), rituxan (CD20 antigen), and ligands such as annexin V (phosphatidylserine) and transferrin (transferrin receptor). Representative aptamer targeting agents include A10 RNA apatamer (prostate-specific membrane antigen) and Thrm-A and Thrm-B DNA apatmers (human alpha-thrombin protein). Targets for the agents noted above are in parentheses. Representative nucleic acid targeting agents include DNAs (e.g., cDNA) and RNAs (e.g., siRNA).

In one embodiment, the invention provides a nanoparticle, comprising:

(a) a core comprising a magnetic material and having a surface;

(b) a coating on the surface of the core, the coating comprising a polyethylenimine and a copolymer comprising a chitosan and a poly(ethylene oxide) oligomer; and

(c) a targeting agent covalently coupled to the coating.

Fluorescent Agents.

Suitable fluorescent agents include fluorescent agents that emit light in the visible and near-infrared (e.g., fluorescein and cyanine derivatives). Representative fluorescent agents include fluorescein, OREGON GREEN 488, ALEXA FLUOR 555, ALEXA FLUOR 647, ALEXA FLUOR 680, Cy5, Cy5.5, and Cy7.

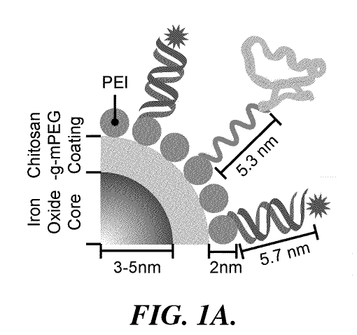

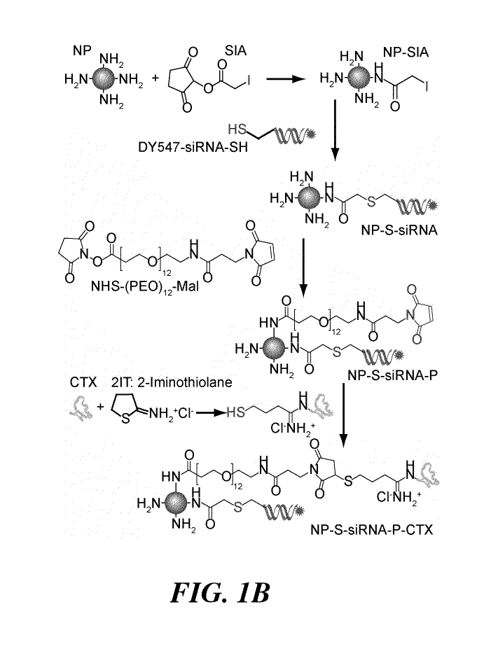

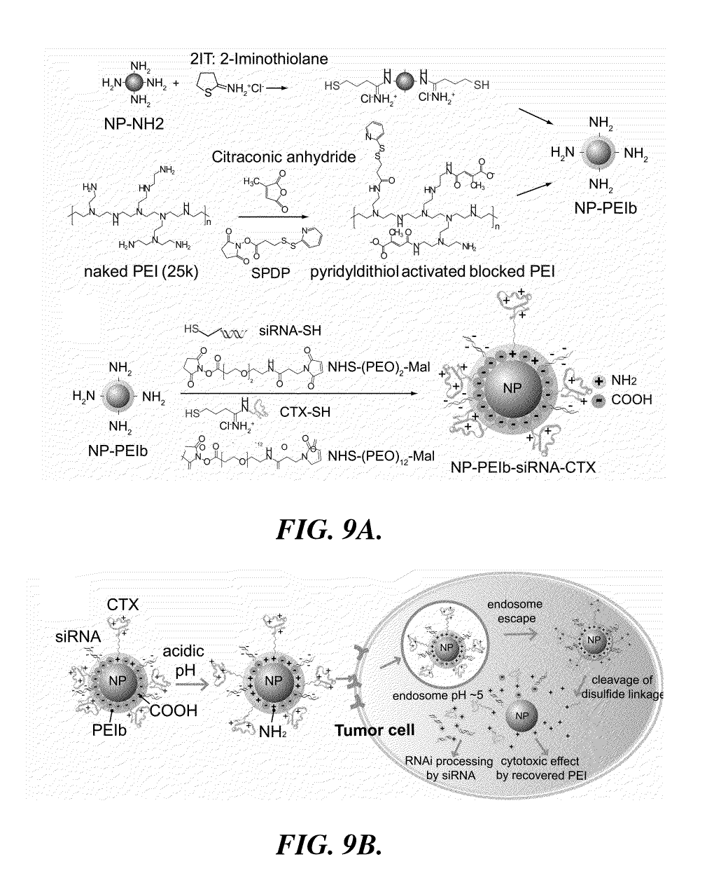

The preparation of representative nanoparticles of the invention is described in Examples 1 and 2 and illustrated schematically in FIGS. 1B and 9A. A schematic illustration of a representative nanoparticle of the invention including a targeting agent (e.g., CTX), a therapeutic agent (e.g., siRNA), and a fluorescent agent is shown in FIG. 1A. A mechanism of action is schematically illustrated in FIG. 9B.

In another aspect of the invention, a composition is provided that includes a nanoparticle of the invention and a carrier suitable for administration to a human subject. Suitable carriers include those suitable for intravenous inject (e.g., saline or dextrose).

In other aspects, the invention provides methods for using the nanoparticles of the invention. The methods include imaging methods such as magnetic resonance imaging when the core has magnetic resonance activity, and optical imaging when the nanoparticle includes a fluorescent agent. The nanoparticles of the invention can also be used for drug delivery when the nanoparticle includes a therapeutic agent. For nanoparticles of the invention that include targeting agents, imaging of and drug delivery to target sites of interest are provided.

In one embodiment, the invention provides a method for detecting (or imaging) cells or tissues by magnetic resonance imaging, comprising:

(a) contacting cells or tissues of interest with a nanoparticle of the invention having affinity and specificity for the cells or tissues of interest, wherein the nanoparticle comprises
(i) a core comprising a magnetic material and having a surface,
(ii) a coating on the surface of the core, the coating comprising a polyethyleneimine and a copolymer comprising a chitosan and a poly(ethylene oxide) oligomer, and
(iii) a targeting agent covalently coupled to the copolymer, wherein the targeting agent has an affinity and specificity to the cells or tissues of interest; and

(b) measuring the level of binding of the nanoparticle to the cells or tissues of interest, wherein an elevated level of binding, relative to normal cells or tissues, is indicative of binding to the cells or tissues of interest.

In the method, the level of binding is measured by magnetic resonance imaging techniques. In a further embodiment of the above method, the nanoparticle further includes a fluorescent agent. In this embodiment, the level of binding can be measured by magnetic resonance and/or fluorescence imaging techniques. The methods are applicable to detecting or imaging cells or tissues in vitro. The methods are also applicable to detecting or imaging cells or tissues in vivo. In this embodiment, the nanoparticles are administered to a subject (e.g., warm-blooded animal) by, for example, intravenous injection.

In another embodiment, the invention provides a method for treating a tissue, comprising contacting a tissue of interest with a nanoparticle of the invention having affinity and specificity for the tissue of interest, wherein the nanoparticle comprises

(a) a core comprising a core material and having a surface,

(b) a coating on the surface of the core, the coating comprising a polyethylenimine and a copolymer comprising a chitosan and a poly(ethylene oxide) oligomer, and

(c) a targeting agent covalently coupled to the copolymer, wherein the targeting agent has an affinity and specificity to the cells or tissues of interest.

In a further embodiment of the above method, the nanoparticle further comprises a therapeutic agent. In this embodiment, the therapeutic agent can be covalently linked (e.g., through a cleavable linkage), physically adsorbed to (e.g., electrostatic or van der Waals interactions), or embedded within the nanoparticle's copolymer coating. The methods are applicable to treating tissues in vitro. The methods are also applicable to treating tissues in vivo. In this embodiment, the nanoparticles are administered to a subject (e.g., warm-blooded animal) by, for example, intravenous injection.

In other aspects, the invention provides methods for silencing or reducing the expression level of a gene. In this embodiment, a cell of interest is contacted with a nanovector of the invention that includes a suitable siRNA.

The following is a description of specific nanovectors of the invention and methods of their use.

In one embodiment, the present invention provides a magnetic nanoparticle platform that includes a superparamagnetic iron oxide (Fe₃O₄) core coated with a cationic copolymer of chitosan-grafted-polyethelyne glycol (PEG) and polyethylenimine (PEI) for non-viral DNA delivery. In this system, the superparamagnetic core enables non-invasive monitoring of DNA delivery in real-time by magnetic resonance imaging (MRI). The combination of chitosan and PEG form a PEGylated chitosan coating on the iron oxide core, stabilizing the nanoparticle from agglomeration. Cationic PEI was incorporated into the coating to serves to associate nucleic acid and to enable proper intracellular trafficking.

In one embodiment, the multifunctional magnetic nanovector of the present invention effectively delivers an amount of siRNA sufficient to target cells to induce gene silencing, while providing the capability of carrier monitoring through MRI. In the nanovector of the invention, siRNA is covalently attached to nanoparticles to prevent its degradation by extracellular or intracellular enzymes, and thus improving the efficacy in gene silencing. To enable site specificity, the targeting peptide chlorotoxin (CTX) was covalently attached to the nanoparticles. CTX is a 4-kDa cationic peptide having a high affinity to the vast majority of brain tumors (74 of 79 World Health Organization classifications of brain tumors) as well as prostate, skin, and colorectal cancers. It is postulated that the target of CTX on cancer cells is associated with the membrane-bound matrix metalloproteinase-2 (MMP-2) protein complex, which is up-regulated on brain tumors and other invasive cancer. The representative nanovector effectively delivers siRNA to brain tumor cells through receptor-mediated endocytosis, and is effective in specific knockdown of the transgene expression of green fluorescence protein (GFP) in C6/GFP+ glioma cells.

Nanovector Containing Low Molecular Weight Polyethylenimine

In this embodiment, the nanovector of the invention includes a low molecular weight polyethylenimine (PEI). The use of low molecular weight PEI imparts the advantageous properties of PEI to the nanovector, while at the same time reducing the disadvantages associated with PEI including toxicity. In this embodiment, the PEI is covalently immobilized on the nanovector via a disulfide linkage that is cleavable after cellular internalization of the nanovector. CTX as a tumor-specific targeting ligand and siRNA as a therapeutic payload are conjugated on the nanovector via a flexible and hydrophilic PEG linker for targeted gene silencing in cancer cells. The nanovector exhibits long-term stability and good magnetic property for magnetic resonance imaging as well as significant cytotoxic and gene silencing effects.

A representative nanovector including low molecular weight polyethylenimine comprises:
(a) a core having a surface and comprising a core material; and
(b) a coating on the surface of the core, the coating comprising
   (i) a copolymer comprising a chitosan and a poly(ethylene oxide) oligomer; and
   (ii) a polyethylenimine having an average molecular weight from about 500 to about 10,000 g/mole;
(c) a targeting agent covalently coupled to the coating; and
(d) an siRNA covalently coupled to the coating.

Nanovector Synthesis.

The preparation of a representative cancer targeting magnetic nanovector (NP-siRNA-CTX) of the invention is described in Example 1 and illustrated in FIG. 1B. The representative nanovector includes a superparamagnetic iron oxide nanoparticle core (6-10 nm diameter) coated with chitosan-g-PEG (2.2 molar ratio of chitosan:PEG. PEI is subsequently attached to the chitosan-g-PEG coating to enable proper intracellular trafficking. PEI is known for its ability to facilitate endosome escape via the “proton sponge effect” wherein the influx of protons and counter-ions into the endosome increases the osmotic pressure, leading to swelling and rupture of endosomes and release of its contents. The base nanoparticle (NP) (i.e., the iron oxide nanoparticles coated with chitosan-g-PEG copolymer and PEI) was synthesized as illustrated in FIG. 1B.

Fluorescently modified siRNA (Dy547-siRNA with 21 nucleotide and 5.7 nm in length), designed to knockdown the transgene expression of green fluorescence protein (GFP), was covalently attached to the PEI on the nanoparticle by reacting its amine groups with thiol-reactive iodoacetyl groups of SIA on NP. A heterobifunctional PEO linker, N-hydroxysuccinimide (NHS)-polyethylene oxide (PEO)₁₂-maleimide of 5.3 nm in length was also attached to the PEI to serve as an anchor for further attachment of the targeting ligand CTX, which could allow CTX to be exposed outwardly and effectively interact with target cells. CTX was first thiolated through reaction with 2-iminoothiolane (2IT), and then conjugated to the PEO linker through the reaction of its thiol group with the malemide group of the PEO, to form the nanovector (NP-siRNA-CTX). A nanovector without CTX (i.e., NP-siRNA) was also prepared as a control. CTX peptides anchored to short chains of PEO can assemble on nanoparticle surfaces as multivalent displays prompting enhanced affinity to cancer cells compared to the free peptide.

Characterization of siRNA Loading and Stability of the Nanovector.

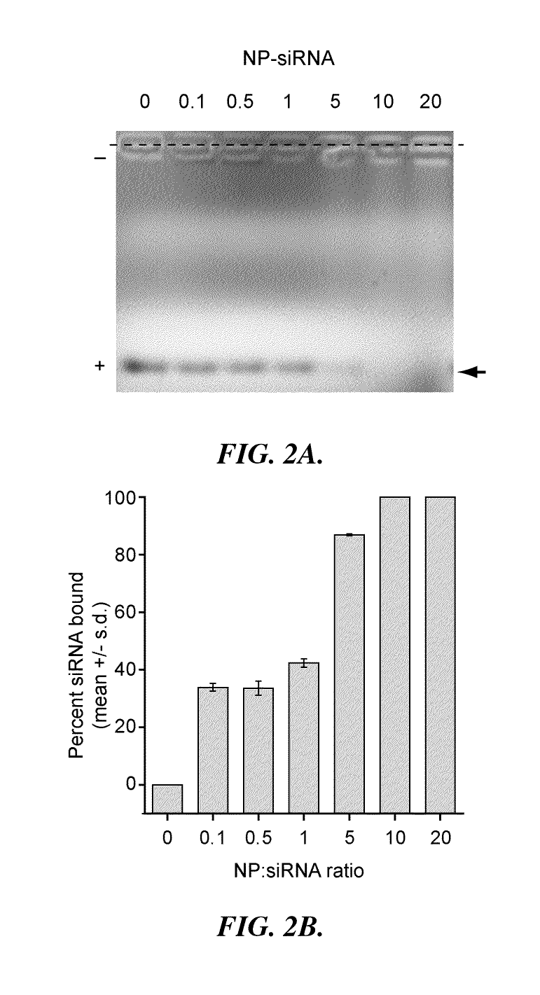

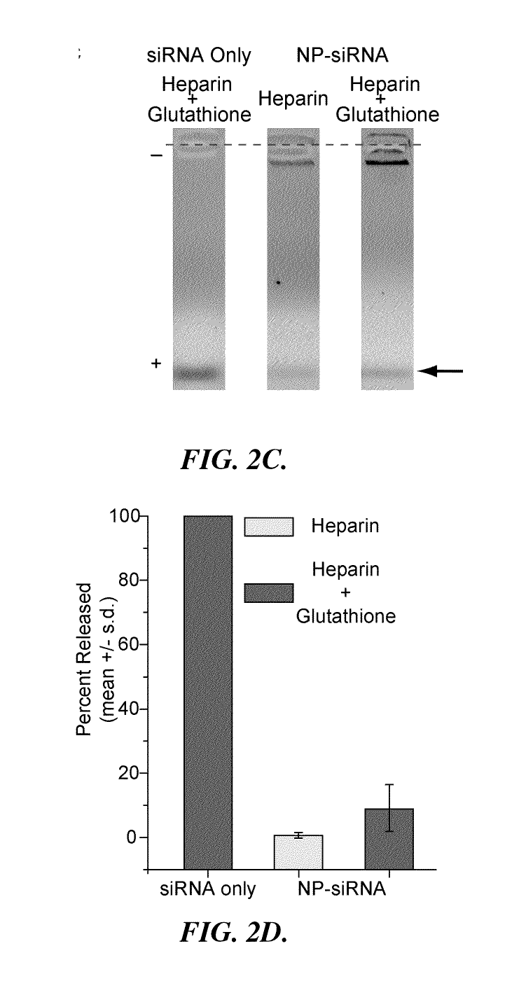

The siRNA loading efficiency of the nanovector was assessed using gel retardation assays (FIGS. 2A-2D). The NP-siRNA-CTX was prepared at varying NP:siRNA weight ratios (Fe mass of NP:siRNA mass) and the reaction time was optimized to allow for the completion of the reaction for each ratio. The reaction products were then loaded in agarose gel wells without purification (FIG. 2A). siRNA bound to NP remained in wells, while unbound siRNA migrated towards the positive electrode (bottom). FIG. 2B shows the corresponding quantitative volume measurements for the intensities of migrating siRNA bands. As shown, at a ratio of 10, 100% of the siRNA was completely bound to NP, and thus this ratio was used for subsequent experiments in this study.

To verify that a stable covalent thioether linkage was formed between NP and siRNA, the release properties of the nanovector after treating with heparin, or heparin+glutathione mixture were evaluated. For these assays, the strongly anionic molecule, heparin, and glutathione, were used as an electrostatic disrupting agent and a reducing agent, respectively, to examine the stability of covalent linkage between siRNA and NP in the presence of exterior competitive molecules and in reductive environments. Naked siRNA was provided as a reference of unbound siRNA. FIG. 2C shows the images of gels analyzed in these assays and FIG. 2D shows the corresponding quantitative results in term of percent siRNA released, as determined by the volume-based intensity measurements of siRNA bands. The results of these assays showed that the NP-siRNA complex exhibited negligible siRNA release in response to either heparin or glutathione treatment confirming the formation of a robust non-cleavable covalent linkage between the NP and siRNA. Covalent attachment of siRNA to a nanocarrier is desirable for in vivo applications because it ensures that the siRNA and carrier will remain intact during blood circulation. A thioether linkage to facilitate covalent conjugation of siRNA to the nanoparticles. To favor thioether bond formation over electrostatic binding of the negatively charged siRNA with cationic NP the reaction was performed in a high ionic strength buffer.

The physicochemical properties of nanoparticles, particularly the hydrodynamic size and zeta potential, are known to greatly influence their behavior both in vitro and in vivo, and internalization by cells. The hydrodynamic size and zeta potential of the nanovector were measured by dynamic light scattering (DLS) and these results are summarized in Table 1.

TABLE 1
Primary physicochemical properties of NP-siRNA-CTX.

| Core | Hydrodynamic | Zeta | | | Magnetic |
|---|---|---|---|---|---|
| Size | Size | Potential | siRNA/ | CTX/ | Relaxivity |
| (nm) | (nm) | (mV) | NP | NP | R₂ (S⁻¹ · mM⁻¹) |
| 7.5 | 111.9 ± 52.4 | +19.6 ± 5.7 | 3.8 | 5 | 214.07 |

Referring to Table 1, the NP-siRNA nanovectors (NP:siRNA ratio of 10:1) have an average core size of 7.5 nm, a hydrodynamic size of 111.9±52.4 nm, and a cationic zeta potential of 19.6±9.7 mV. The number of siRNA and CTX molecules per nanoparticle were calculated to be 3.8, and 5 respectively, as determined by gel retardation assays. The magnetic relaxivity of each NP-siRNA, which determines the detectability of the magnetic nanovectors by MRI, was evaluated by R₂ measurements, and was found to be 214.07 S⁻¹ mM⁻¹. The hydrodynamic size of NP-siRNA falls within the acceptable size range (5<d<200 nm) to ensure in vivo success and evasion of sequestration by macrophages.

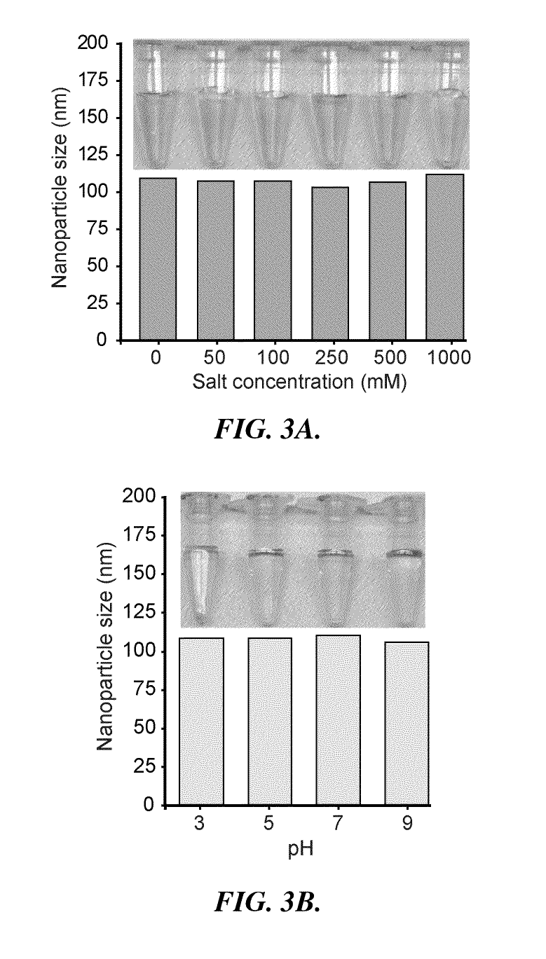

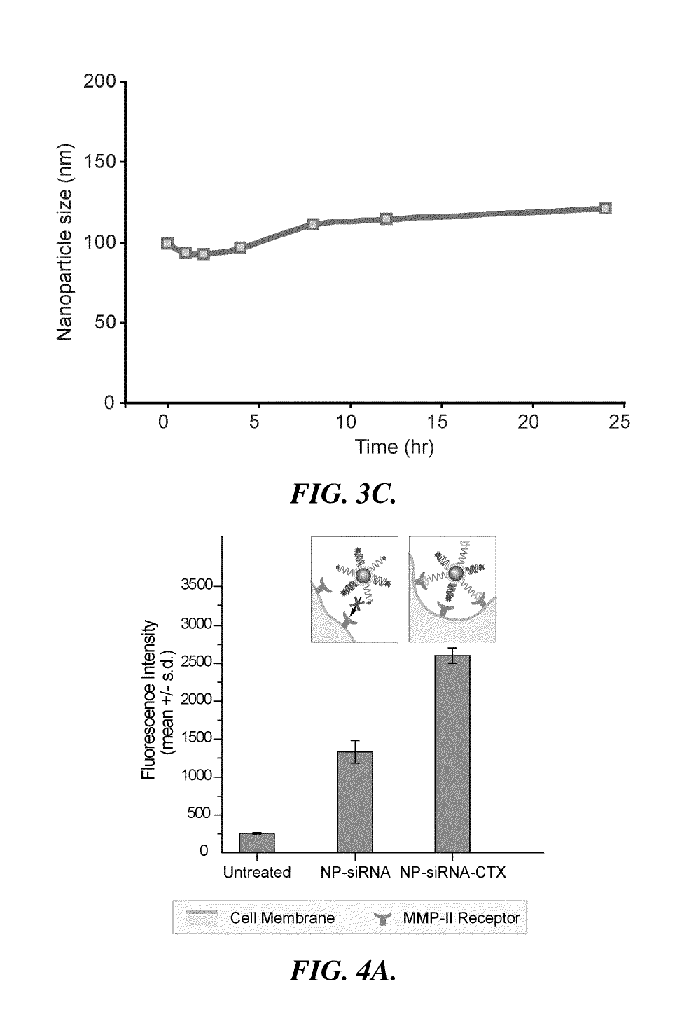

The stability of the nanovector in biological media, which is critical to their clinical success, was evaluated by monitoring their hydrodynamic size changes in solutions over a range of pH and salt concentrations, and in DMEM cell culture media containing 10% FBS at 37° C. over a 24-hour period (FIGS. 3A-3C). The results showed that no appreciable change in the hydrodynamic size of nanovectors was observed in response to increased NaCl concentrations over 0-1000 mM (FIG. 3A) or to pH changes from 3 to 9 (FIG. 3B). This can also be visualized by images shown in FIGS. 3A and 3B, which show transparent nanovector suspensions with no sign of color change, agglomeration, or precipitation. Similarly, the hydrodynamic sizes of nanovectors in the cell culture media remained approximately constant over the 24-hour period (FIG. 3C). Combined, these colloidal stability tests confirm that the NP-siRNA construct is stable under biologically relevant conditions.

The colloidal stability in biological environments can be a challenging issue in clinical use of any nanoparticle-based constructs due to the large surface area to volume aspect ratio of nanoscale materials. Charged cationic nanoparticles, in particular, often display poor stability in cell culture conditions, as they tend to adsorb proteins from the biological environment through electrostatic interaction, causing fouling or precipitation. Iron oxide nanoparticles coated with PEI or other cationic polymers are greatly susceptible to aggregation in cell culture environments. The nanovectors of the invention have integrated PEG to prevent non-specific absorption of proteins. Hydrophilic PEG coatings have been shown to resist protein absorption and provide steric hindrance preventing nanoparticles from aggregation. The nanovectors have PEG incorporated in both the initial shell coating and the outer layer exposed to solution environment. These combined measures contribute to the enhanced stability of the nanovector construct, even over the NaCl and pH ranges tested.

Receptor Mediated Internalization of Nanovectors by Cancer Cells.

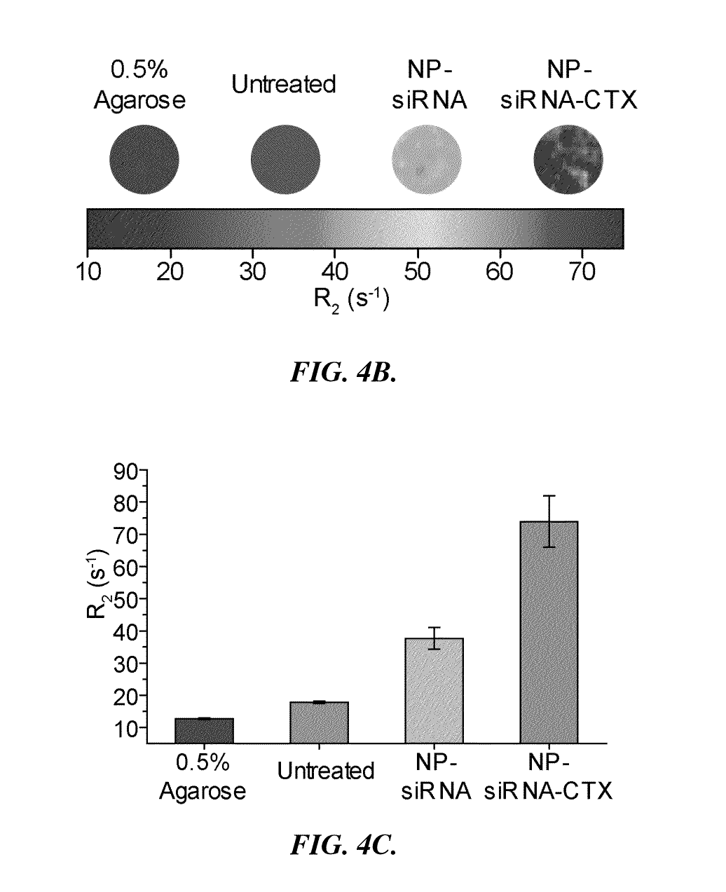

To evaluate the enhanced affinity of our CTX targeted nanovectors to tumor cells, C6 cells were treated with either NP-siRNA-CTX or NP-siRNA (control), and their internalization by the cells were assessed with flow cytometry and MRI. For these experiments, C6/GFP+ cells were treated with 20 μg of Fe/ml (2 μg of siRNA/ml) of NP-siRNA or NP-siRNA-CTX for two hrs prior to analysis. FIG. 4A shows the results of the flow cytometry assay, which monitored Dy547 fluorochrome attached to the siRNA. As shown, NP-siRNA-CTX was internalized by cells 2-fold more than NP-siRNA. As a reference, the mean intensity of untreated cells is also plotted. The diagrams above the graph in FIG. 4A comparatively illustrate how the surface chemistry of each nanovector regulates the internalization of the nanoparticles. NP-siRNA does not bind MMP-2 receptors expressed by the cancer cells (shown as claws), while NP-siRNA-CTX readily binds to glioma cells through the affinity of CTX to MMP-2.

This preferential cell binding for NP-siRNA-CTX was further demonstrated by MRI phantom imaging through the inherent magnetic properties exhibited by the iron oxide core of the nanovector. FIG. 4B shows R₂ maps that highlight the degree of contrast enhancement produced due to the internalization of nanovectors by cells and FIG. 4C shows the corresponding R₂ values measured from each sample. The cells treated with NP-siRNA-CTX produced the highest signal enhancement, which was 4.2 fold higher than those of untreated cells, and 2 fold higher than those of cells treated with NP-siRNA. Both the flow cytometry assay and MRI experiments indicated that CTX modification of nanovector promoted receptor-mediated internalization yielding improved delivery of siRNA to cancer cells. These findings indicate that while some of nanoparticles are able to enter cells through adsorptive mediated endocytosis, it is evident that a substantial enhancement in the cellular internalization of nanovectors and MRI contrast can be achieved through targeting with CTX. The results also indicate that the magnetic properties possessed by the nanovector potentially allow for clinical treatment monitoring through MRI detection.

Nanovector Escape from Endosome Compartments.

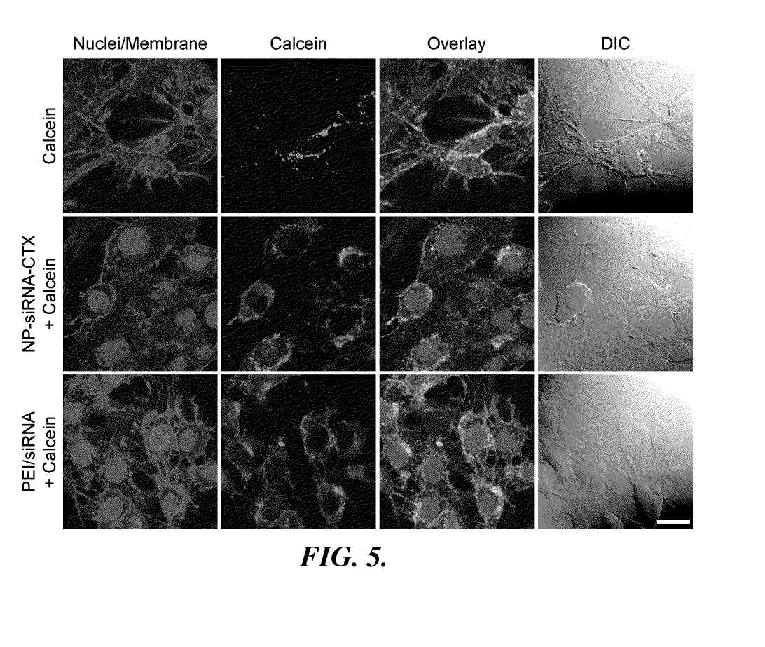

Upon internalization of siRNA carriers by cancer cells, the nanocarrier, along with its siRNA cargo, becomes localized in endosomal compartments and have no access to the cytoplasm to initiate the RNAi pathway. PEI is incorporated in the nanovectors of the invention to enable the nanovector to escape the endosome through the proton sponge effect. An endosomal integrity assay was used to assess the ability of nanovectors to escape endosomal compartments. In this assay, a membrane impermeable fluorescent dye, Calcein (0.25 mM), was delivered alone or co-delivered along with NP-siRNA-CTX or PEI/siRNA complexes (commercial transfection reagent) to C6 cells, and the treated cells were maintained for 2 hrs under normal cell culture conditions. The intracellular localization of calcein was then visualized using fluorescence microscopy with DAPI (cell nuclei) and WGA-647 (cell membranes) counterstaining (FIG. 5). As shown in cells treated with calcein alone, the membrane impermeable calcein did not spread throughout cells indicating the integrity of these endosomes. Conversely, in cells co-treated with calcein and NP-siRNA-CTX or PEI/siRNA nanovectors, calcein was distributed throughout the cells, which indicates the disruption of the endosomes and release of calcein into the cytoplasm of the cells. This visual observance verifies that NP-siRNA-CTX is able to promote endosomal escape similarly to PEI/siRNA.

Receptor Mediated Gene Knockdown and Monitoring of Toxicity.

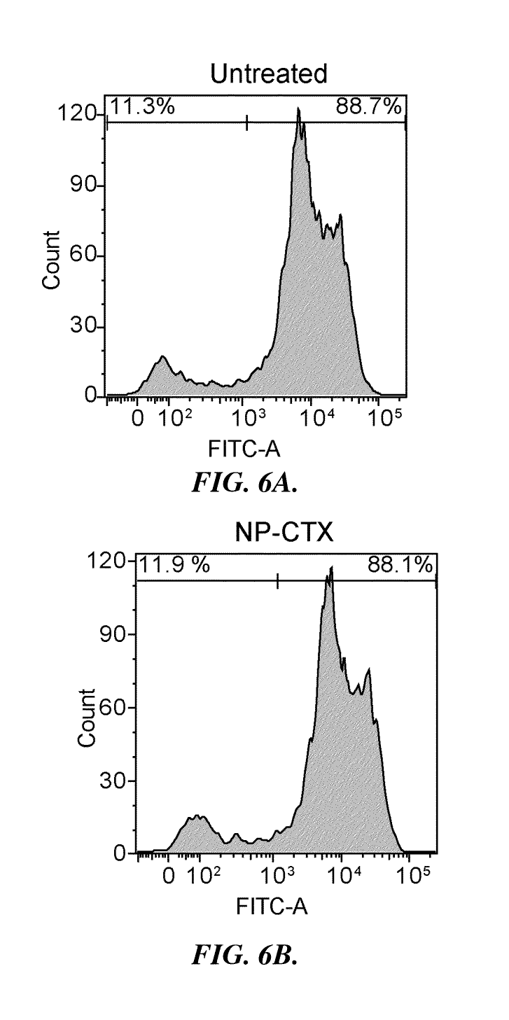

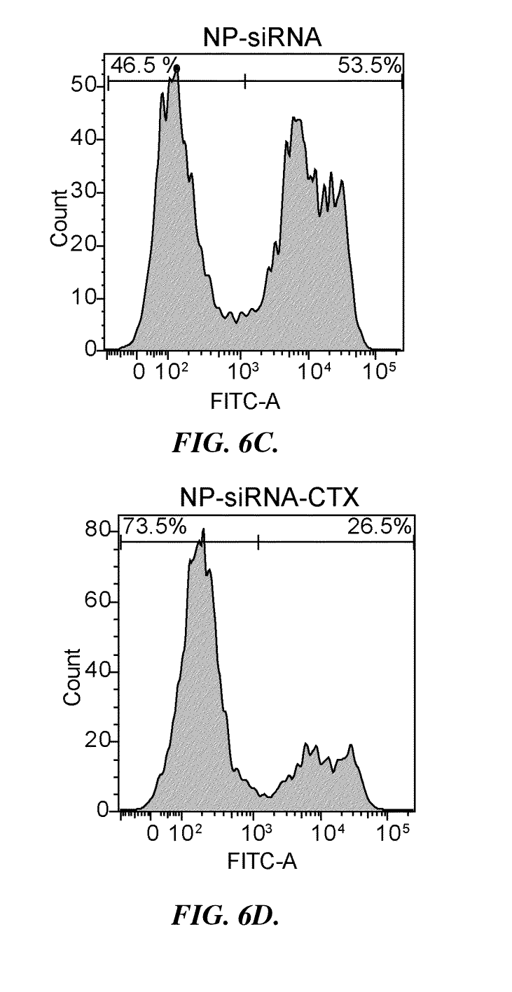

After confirming that the nanovector was able to promote endosomal escape, the ability of the nanovector to knockdown the transgene expression of GFP in C6/GFP+ cells was evaluated after the cells were treated with either NP-siRNA or NP-siRNA-CTX. A treatment of cells with NP-CTX was used as negative control nanoparticles (i.e., targeting nanoparticles that carry no siRNA) to confirm that NP and CTX do not have an effect on GFP expression. C6/GFP+ cells were treated with each nanovector at a concentration of 2 μg/ml of siRNA for two hrs, then analyzed using flow cytometry 48 hrs post treatment. FIGS. 6A-6_ show the percentage of GFP expressing cells post treatment. The GFP expression profile of untreated C6/GFP+ cells, serving as a baseline reference, indicates that 88.7% of the cells were GFP positive (FIG. 6A). Cells treated with NP-CTX (FIG. 6B) showed no appreciable change in GFP expression compared to untreated cells (88.1% versus 88.7%), which confirms that NP and CTX did not affect GFP expression, as expected. Cells treated with NP-siRNA (FIG. 6C) exhibited a 35% reduction in GFP expression (53.5% versus 88.7%). Significantly, cells treated with NP-siRNA-CTX treatment (FIG. 6D) showed a 62% reduction in GFP expression compared to untreated cells (26.5% versus 88.7%). These results indicate that through the receptor-mediated cellular internalization of NP-siRNA-CTX, enhanced knockdown of GFP expression in C6/GFP+ cells was achieved.

Non-targeting cationic vectors can be internalized by cells non-specifically through adsorptive mediated endocytosis. However, prior studies have also shown this mechanism of cellular internalization is not as effective as targeted delivery and yet can only be used to treat a certain percentage of the cellular population. The present invention provides targeted delivery and ensures that a greater percentage of the cell receives an effective dose of siRNA.

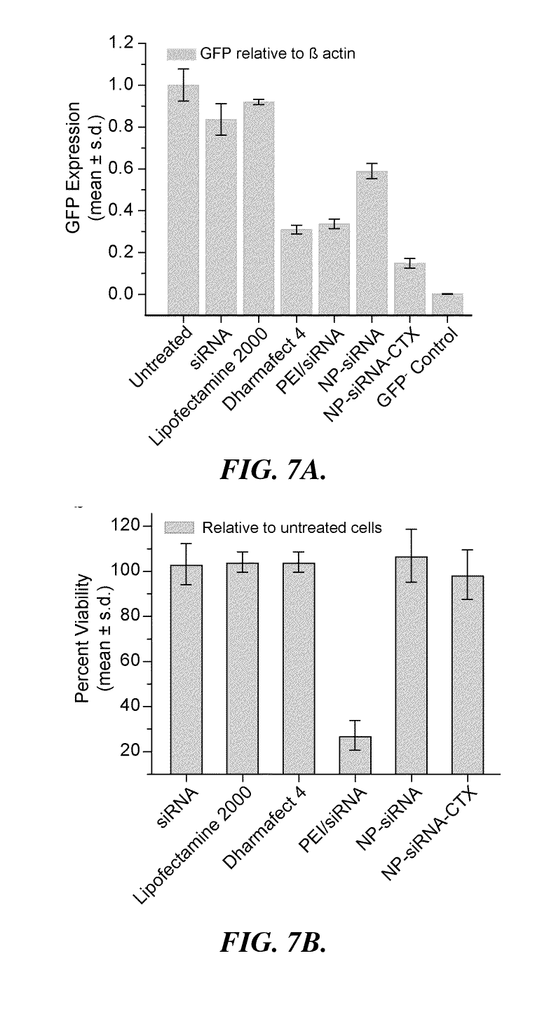

The targeted siRNA delivery and enhanced gene knockdown by the NP-siRNA-CTX nanovector of the invention was demonstrated through quantitative RT-PCR analysis (FIGS. 7A and 7B). In these experiments C6/GFP+ cells were treated with nanovectors as described above. For comparison, the efficacies of the following additional gene transfection reagents: naked siRNA, NP-siRNA, PEI/siRNA, Dharmafect 4, and Lipofectamine 2000 were evaluated. PEI/siRNA is a commonly used cationic complex with high gene transfection efficiency, and Dharmafect 4 and Lipofectamine 2000 are two of the most widely used commercial gene transfection reagents. Cells were treated with one of these reagents and analyzed 48 hrs after treatment. As negative control, non-transfected C6 cells (GFP−) were also assayed to obtain a reference for cells with zero GFP expression.

FIG. 7A shows the GFP mRNA transcripts of cells for each treatment relative to the housekeeping gene β-actin. NP-siRNA treatment reduced GFP mRNA transcripts to 0.58 relative to those in untreated cells, representing a 1.7 fold knockdown in gene expression. NP-siRNA-CTX treatment reduced GFP mRNA levels to 0.23 relative to those in untreated cells, a 4.3-fold knockdown in gene expression. These results are consistent with those obtained from the flow cytometry experiments. By comparing the transfection efficiencies of these two nanovectors, a 2.5-fold enhancement in gene knockdown was observed as a result of the targeted delivery by incorporation of CTX in the nanovector. Cells treated with naked siRNA or Lipofectamine 2000 did not show significant GFP expression knockdown, and PEI/siRNA and Dharmafect 4 treatments were able to knockdown GFP expression by about 3 fold. NP-siRNA-CTX nanovector was most efficient among all transfection reagents evaluated and highlights its advantage as a targeted delivery system.

FIG. 7B shows the cell viability after cells were treated with each transfection reagent. It is seen that only PEI/siRNA treatment generated significant cytotoxicity. The cytotoxicity exhibited by PEI/siRNA can be attributed to the presence of unmodified PEI whose toxicity is well documented. The toxic effect of PEI in NP-siRNA-CTX (NP-siRNA as well) was modulated by inclusion of PEG in the nanovector of the invention. This modification shields the cationic nature of PEI and thus reduces (or eliminates) toxicity associated with PEI.

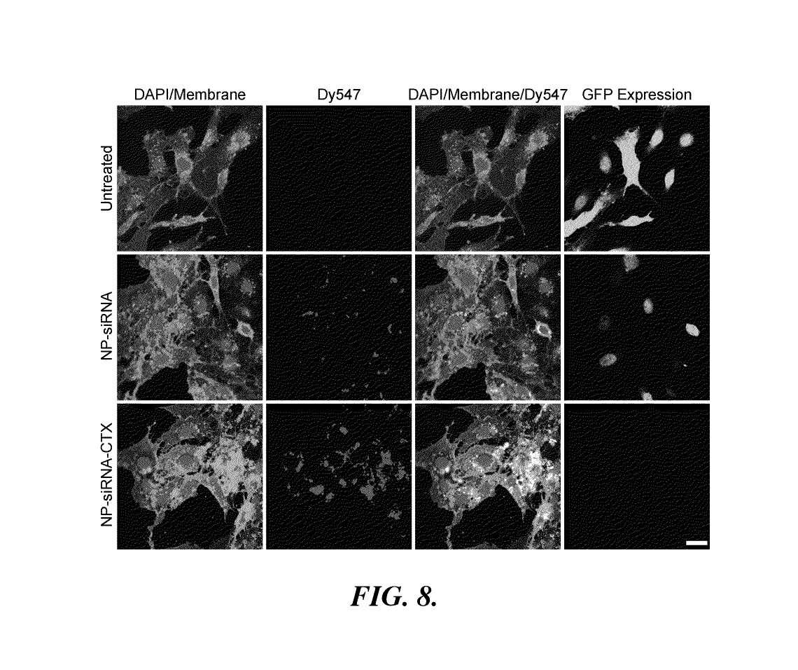

To visualize the targeted delivery of siRNA and enhanced knockdown of gene expression by NP-siRNA-P-CTX, cells treated with either NP-siRNA-CTX or NP-siRNA were examined by confocal fluorescence microscopy (FIG. 8). In these fluorescence images, cell nuclei were stained with DAPI (blue) and membranes with WGA-647 (green). The images of untreated cells are provided as a reference. Treatments with NP-siRNA and NP-siRNA-CTX were administered as described above. FIG. 8 shows that the relative amount of siRNA (Dy547, second column) internalized by cells treated with NP-siRNA-CTX is significantly higher than in cells treated with NP-siRNA. The overlay images (third column) reveal that the delivered siRNA molecules are predominantly localized in the perinuclear region of cells, where siRNA molecules are recognized by the RISC complex. This observation confirms the proper trafficking of siRNA within cells. The GFP expression analysis (light green, fourth column) showed that the NP-siRNA treatment reduced the GFP expression of cells compared to the untreated cells, but GFP+ cells were still noticeably present. For cells treated with NP-siRNA-CTX, little to no GFP expression was observed. This fluorescence analysis confirmed the trends demonstrated by flow cytometry and qRT-PCR analyses.

pH-Sensitive Nanovector

In another embodiment, the invention provides a pH-sensitive nanovector. In this embodiment, at least a portion of the primary amine groups of the nanovector's polyethylenimine are converted to amide groups (e.g., capped). In one embodiment, at least a portion of the primary amine groups of the polyethylenimine (PEI) are blocked with citraconic anhydride that is removable at acidic conditions. Capping at least a portion of the primary amine groups not only to increases the nanovector's biocompatibility at physiological conditions, but also elicits a pH-sensitive cytotoxic effect in the acidic tumor microenvironment. In this embodiment, the PEI is covalently immobilized on the nanovector via a disulfide linkage that is cleavable after cellular internalization of the nanovector. CTX as a tumor-specific targeting ligand and siRNA as a therapeutic payload are conjugated on the nanovector via a flexible and hydrophilic PEG linker for targeted gene silencing in cancer cells. With a size of about 60 nm, the nanovector exhibits long-term stability and good magnetic property for magnetic resonance imaging. The multifunctional nanovector exhibits both significant cytotoxic and gene silencing effects at acidic pH conditions for C6 glioma cells, but not at physiological pH conditions.

A representative nanovector having pH-sensitive properties comprises:
(a) a core having a surface and comprising a core material;
(b) a coating on the surface of the core, the coating comprising
   (i) a copolymer comprising a chitosan and a poly(ethylene oxide) oligomer; and
   (ii) a polyethylenimine having primary, secondary, and tertiary amine groups, wherein at least a portion of primary amine groups are modified to amide groups;
(c) a targeting agent covalently coupled to the coating; and
(d) an siRNA covalently coupled to the coating.

Preparation and Characterization of Amine Group Blocked PEI.

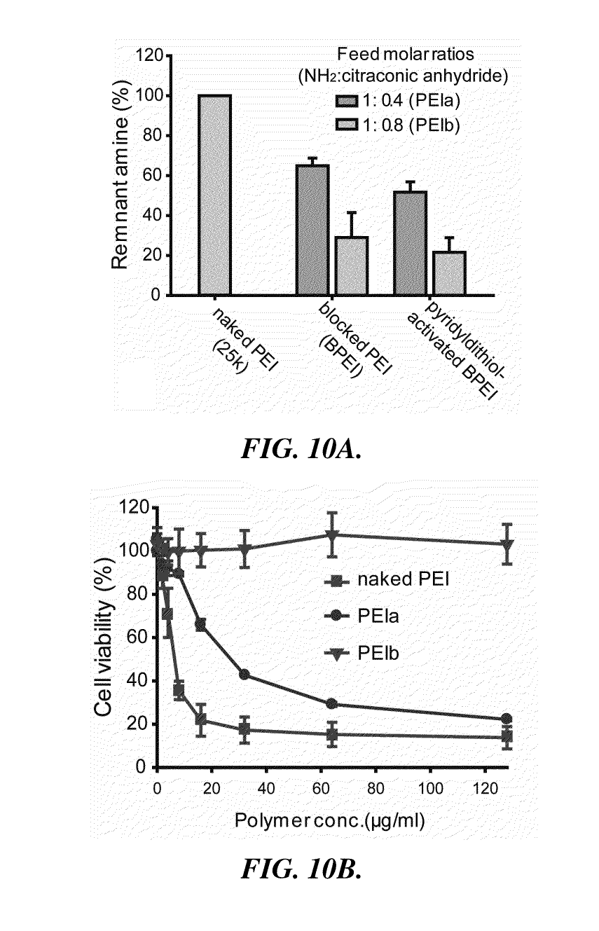

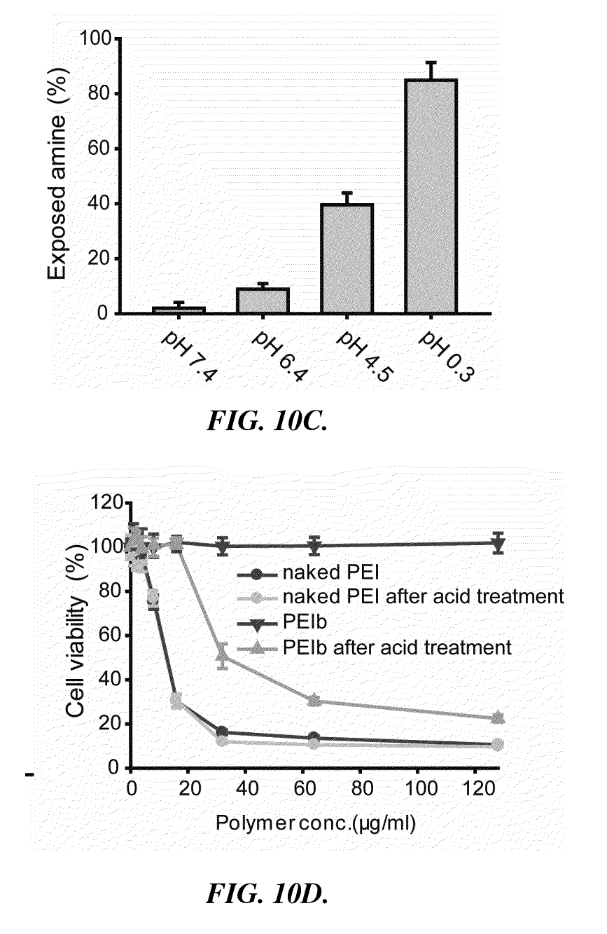

The preparation of a primary amine blocked PEI and a nanoparticle containing the PEI is described in Example 2. The scheme for synthesis of primary amine blocked PEI (25k) is shown in FIG. 9A. The primary amine group of PEI was blocked by citraconic anhydride, an acid pH specific amine blocker. After citraconylation reaction, the primary amine groups are replaced with carboxyl groups, which changes the total cationic charge in PEI. The remaining amine groups in PEI are then activated with SPDP to produce 2-pyridyl disulfide activated PEI for the following conjugation onto iron oxide nanoparticles (NPs). After each reaction, the amount of remaining amine groups was quantitatively examined using the fluorescamine assay. As shown in FIG. 10A, two different types of amine blocked PEI, less amine blocked PEI (PEIa) and highly amine blocked PEI (PEIb), were prepared by adding different amounts of citraconic anhydride. Molar fractions of primary, secondary, and tertiary amine groups in PEI were 0.307, 0.395, and 0.297, respectively. Based on this, the molar ratios of citraconic anhydride per primary amine group in PEI for the preparation of PEIa and PEIb were 0.42:1 and 0.84:1, respectively. After citraconylation, 64.9±3.8 and 28.9±12.4% of primary amine groups in PEIa and PEIb remained, respectively. Approximately 10% of amine groups were also activated by SPDP in both PEIs.

To examine whether blocking of primary amine groups in PEI could reduce nonspecific cytotoxicity, cell viability was examined after treated with naked PEI, PEIa, and PEIb (FIG. 10B). Both naked PEI and PEIa treated cells showed severe cell toxicity with increasing PEI concentrations, while the PEIb group demonstrated negligible cytotoxic effect. To confirm the removal of blocking groups in PEI in an acidic pH-dependent manner, the amount of exposed amine in PEIb was quantitatively examined after incubating PEIb at different pHs (FIG. 10C). The extent of exposed primary amine groups in PEIb at pH 7.4, 6.4, and 4.5 were 1.9±2.1, 8.8±2.0, and 39.5±6.4%, respectively, demonstrating that the deblocking of the primary amine groups occurs only in acidic pH conditions.

To confirm that the deblocking of primary amine groups in PEIb in acidic conditions could recover cell toxicity comparable to naked PEI, cytotoxicities by naked PEI and PEIb before and after acid treatment were examined (FIG. 10D). The PEIb treated with acid treatment exhibited significantly increased cytotoxicity compared to PEIb at pH 7.4, suggesting that the cytotoxicity of this polymer can be elicited by an acidic environment. However, the extent of cytotoxic effect by PEIb treated with acid was a little reduced than that by naked PEI, which is probably due to the noncleavable blocking of primary amine groups in PEI by SPDP.

Preparation and Characterization of Multifunctional Iron Oxide Nanovectors.

The pH-sensitive PEIb useful in the present invention was used for the surface modification of the NP—NH₂ (FIG. 9A). Primary amine groups on NP were reacted with Traut's reagent to produce free thiol modified NP(NP—SH). The pyridyldithiol activated PEIb prepared by SPDP reaction was added to thiol modified NP, which resulted in blocked PEI coated NP(NP-PEIb). The chemical scheme used to assemble siRNA and chlorotoxin (CTX) on the NP surface are shown in FIG. 9B. The thiol-modified siRNA at the sense 5′ end was conjugated to the free amine groups available on PEIb and NP using SM(PEG)₂ as a crosslinker. For enhanced intracellular delivery of the nanovector for glioma cells, CTX was also immobilized to the NP-PEIb-siRNA conjugate. Chlorotoxin, a 36 amino-acid peptide derived from scorpion Leiurus quinquestriatus, is a well-known targeting ligand for malignant glioma, medulloblastoma, and prostate cancers. Thiol-modified CTX by Traut's reagent was covalently attached to NP-PEIb-siRNA conjugate via SM(PEG)₁₂ crosslinker for the preparation of NP-PEIb-siRNA-CTX.

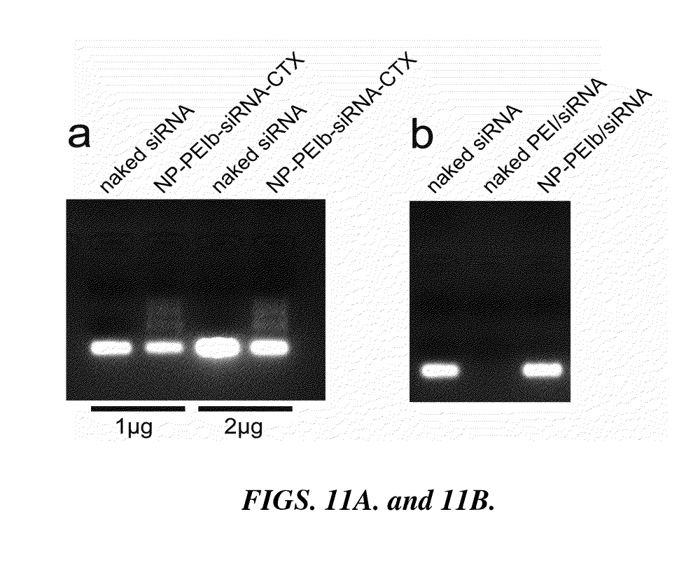

To quantify and optimize the extent of conjugated siRNA to NP, the NP-PEIb-siRNA-CTX nanovector was assessed by agarose gel electrophoresis (FIG. 11A). The siRNA conjugated to NP-PEIb-siRNA-CTX showed a retarded and smear band on agarose gel because of the increased size of the resulting nanovector compared to naked siRNA as well as varying numbers of attached siRNA on individual nanoparticles. The relative amount of siRNA conjugated to nanoparticles for 1 μg and 2 μg siRNA were 37.6±7.4% and 28.9±7.8% of total feed siRNA, respectively, as determined by densitometry using ImageJ software (National Institute of Health, USA; http://rsb.info.nih.gov/ij/). To confirm that this retarded migration of NP-PEIb-siRNA-CTX is not attributed to ionic interaction between siRNA and PEIb, a physical mixture of siRNA and NP-PEIb at the same PEIb/siRNA weight ratio as that of the NP-PEIb-siRNA-CTX conjugates was also examined by gel electrophoresis. As shown in FIG. 11B, NP-PEIb could not form ionic complexes with siRNA while naked PEI/siRNA complexes showed no migration due to ionic interactions, suggesting that the retarded migration of siRNA in NP-PEIb-siRNA-CTX nanovector is due to the covalent linkage of siRNA to NP rather than the electrostatic interaction between PEIb and siRNA.

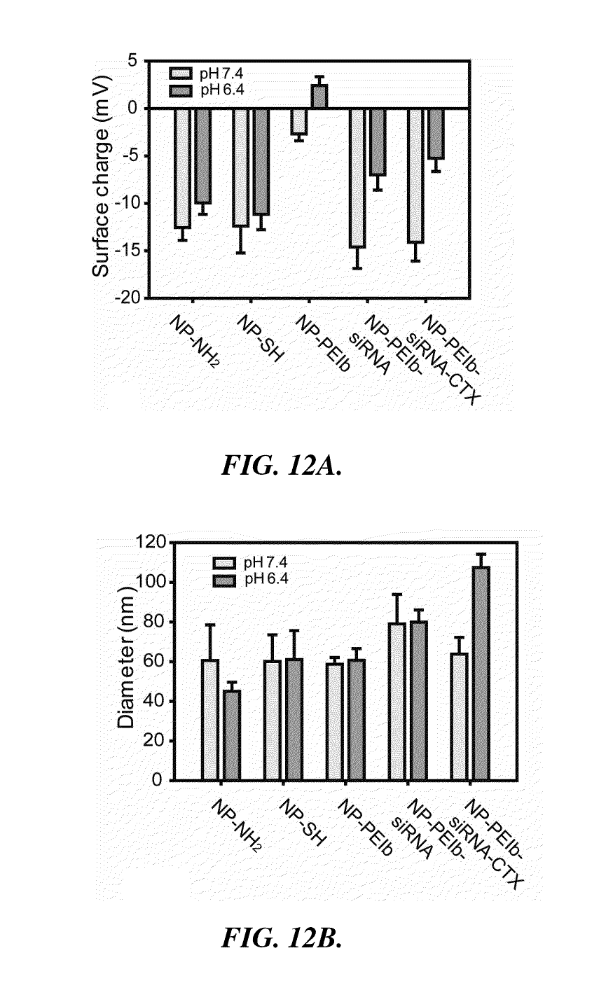

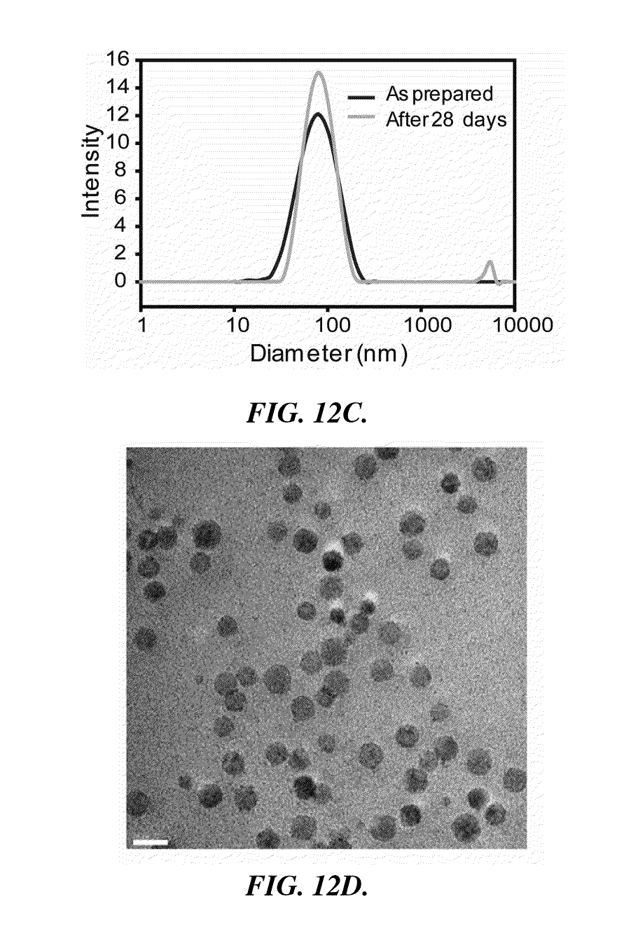

The average surface charge and hydrodynamic size of the resulting five different types of nanoparticles were determined by dynamic light scattering (DLS). (FIGS. 12A and 12B). Conjugation of PEIb onto NP—SH increased surface charge from −12.4±2.8 mV to −2.6±0.7 mV at pH 7.4 due to both remnant primary amine groups and intact secondary and tertiary amine groups. However, after siRNA conjugation, the surface charge of nanoparticles decreased to −14.6±2.2 mV because of the highly negatively charged siRNA. The surface charges of NP-PEIb-siRNA-CTX at pH 7.4 and pH 6.4 were −14.1±1.9 mV and −5.2±1.3 mV, respectively. Considering that there were ˜174 primary amine group per every PEI, a 10% recovery of amine groups at pH 6.4 could substantially increase the surface charge. The hydrodynamic sizes of each type of nanoparticles at pH 7.4 and pH 6.4 are shown in FIG. 12B. While there were significant changes in surface charge after conjugation of PEIb and siRNA, little differences in particle size were observed among NP, NP—SH, NP-PEIb, and NP-PEIb-siRNA. Despite the significant differences in surface charge at different pHs, the size of each nanoparticle was pretty constant, which might be due to sufficient PEG shielding on nanovector. However, the diameter of NP-PEIb-siRNA-CTX at pH 6.4 (107.4±6.7 nm) was significantly larger than that at pH 7.4 (63.7±8.4 nm) (FIG. 12B). Considering that the pore size of 1% agarose gel was more than 350 nm, the nanoparticles with a size below 70 nm could easily pass through the agarose gel as shown in FIG. 11A.

For biomedical applications, long-term stability of nanoparticles is an important factor considered during design of nanoparticles. To examine the stability of NP-PEIb-siRNA-CTX, size distributions of nanoparticles both as prepared and after 28 days in PBS solution were analyzed. As shown in FIG. 12C, negligible changes in particle size were observed between the two, suggesting the high stability of NP-PEIb-siRNA-CTX. The core particle size and morphology of NP-PEIb-siRNA-CTX was examined with TEM (FIG. 12D). NP-PEIb-siRNA-CTX was spherical and well-dispersed, with a core size of about 12 nm, indicating that there was no aggregation or inter-crosslinking between particles during conjugation reaction.

Magnetic Property and Intracellular Uptake of NP-PEIb-siRNA-CTX.

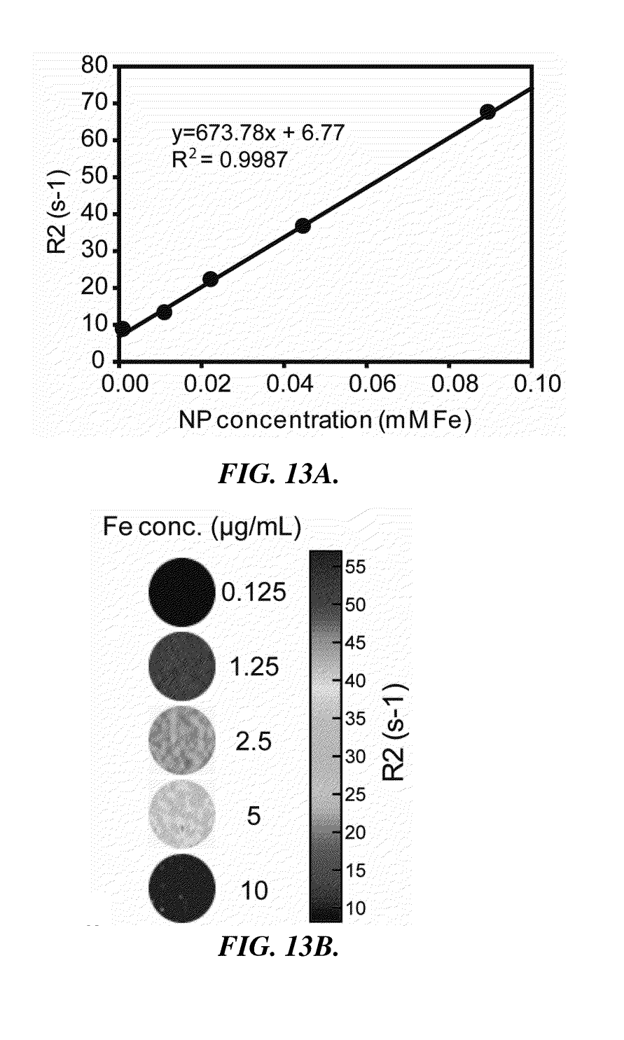

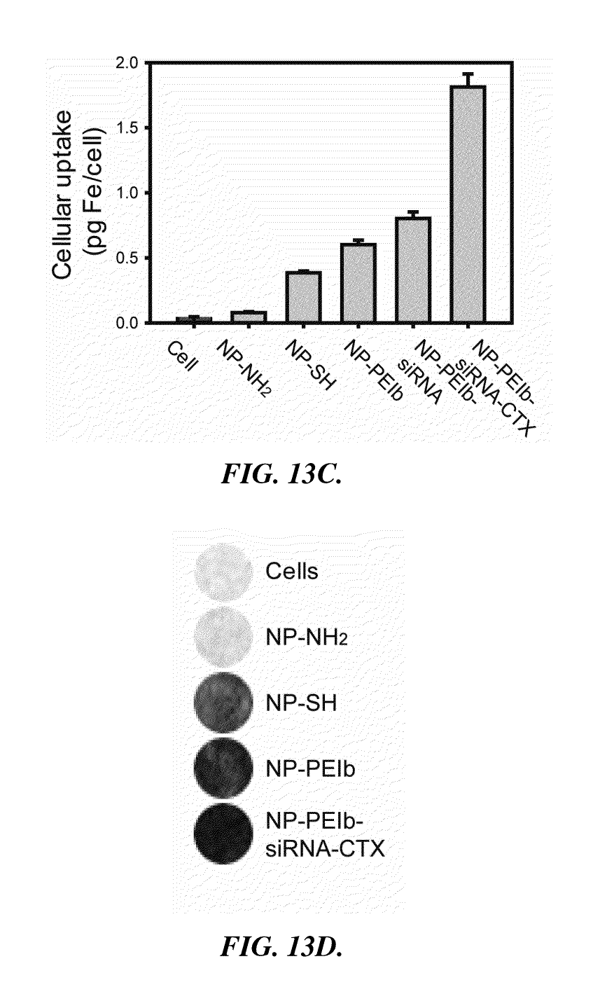

The iron oxide core of the NP system possesses superior superparamagnetic properties essential for use as a contrast agent for MR imaging. To evaluate whether NP-PEIb-siRNA-CTX would retain sufficient magnetism detectable by MRI, the relaxation of NP-PEIb-siRNA-CTX at various concentrations of Fe was measured. The transverse relaxivity (slope of R2) of NP-PEIb-siRNA-CTX was 673 mM⁻¹ S⁻¹ (FIG. 13A), significantly higher than that of Feridex (˜230 mM⁻¹ S⁻¹), a commercial T2 contrast agent, making NP-PEIb-siRNA-CTX potentially a very effective T2 contrast agent for MRI. FIG. 13B shows that relaxation (R2) changes of agarose phantom casts with NP-PEIb-siRNA-CTX with increasing Fe concentration, could be readily detected by MRI in a dose dependent manner.

To confirm that conjugation of CTX could enhance intracellular uptake of nanoparticles, the amount of intracellular Fe per cell was quantified using a ferrozine-based assay after treating C6 cells with five different nanoparticles at an Fe concentration of 4 μg/mL for 6 hrs. As shown in FIG. 13C, NP-PEIb-siRNA-CTX exhibited 2-4 fold higher Fe uptake than NP—SH, NP-PEIb, and NP-PEIb-siRNA at pH 6.4. The enhanced intracellular uptake of NP-PEIb-siRNA-CTX over that of NP-PEIb-siRNA despite the similar surface charge exhibited by the two nanovectors suggests that the immobilization of CTX facilitated the cellular internalization of the nanovector. This enhanced cellular uptake was also visualized by T2-weighted MR images of cells incubated with different nanoparticles (FIG. 13D). Cells incubated with NP-PEIb-siRNA-CTX generated the darkest image compared to those treated with NP—NH₂, NP—SH, or NP-PEIb.

Cell Toxicity and Gene Silencing Effect by NP-PEIb-siRNA-CTX.

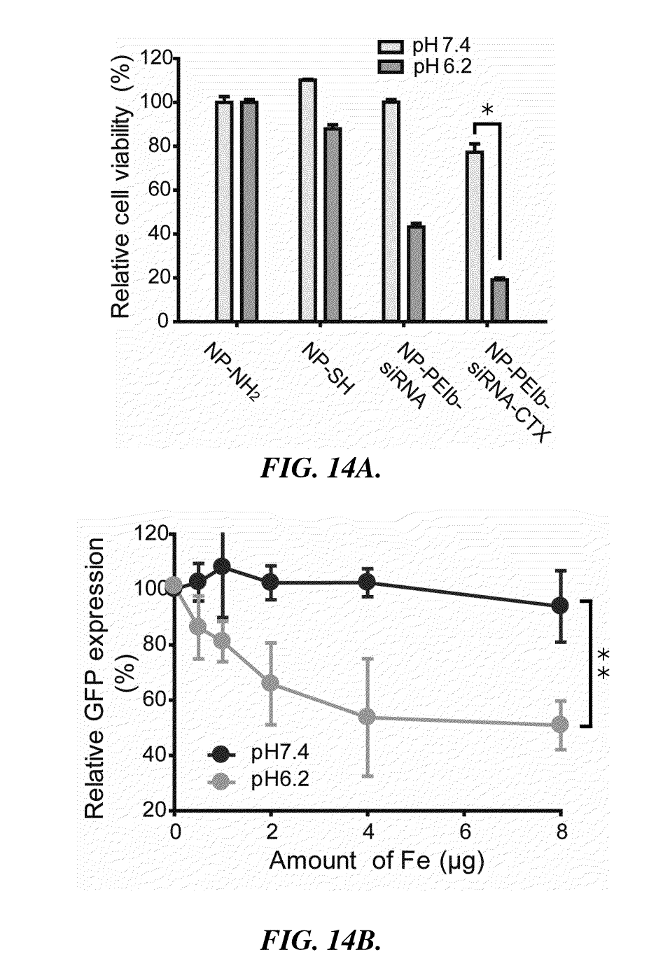

FIG. 14A shows the elicited cytotoxic effect in C6 cells treated with four different types of nanoparticles at both pH 7.4 and pH 6.2. While no significant cytotoxicity was elicited by any of these nanoparticles at pH 7.4, nanoparticles coated with PEIb (i.e., NP-PEIb-siRNA and NP-PEIb-siRNA-CTX) exhibited greatly enhanced cytotoxic effect at pH 6.2, indicating that nanoparticles coated with amine group-blocked PEI induced pH-specific cell-killing for cancer cells. NP-PEIb-siRNA elicited significant cytotoxicity at pH 6.2 but not at all at pH 7.4. The viabilities of cells treated with NP-PEIb-siRNA-CTX at pH 7.4 and pH 6.2 were 77.2±3.7% and 19.1±0.8%, respectively. There was no cytotoxic effect on C6 cells by acidic pH itself down to pH 5.4 (data not shown). This is likely due to cancer cells having their own mechanism of survival in acidic extracellular conditions.

Gene silencing effects by NP-PEIb-siRNA-CTX were evaluated at both pH 7.4 and pH 6.2 in the presence of 10% serum (FIG. 14B). The extent of GFP gene expression by cells treated with NP-PEIb-siRNA-CTX at pH 6.2 was significantly decreased with increasing amount of NP-PEIb-siRNA-CTX, while little change in GFP gene expression was observed in cells receiving the same treatments at pH 7.4. As shown in FIG. 12A, the negative surface charge on NP-PEIb-siRNA-CTX at pH 7.4 is nearly 3 times greater than that at pH 6.2. This highly negatively charged nanovector at pH 7.4 could hinder its cellular internalization, resulting in substantially-reduced gene silencing. The relative percentages of GFP gene expression after transfecting C6 cells with 8 μg of NP-PEIb-siRNA-CTX at pH 7.4 and pH 6.2 were 93.8±12.9% and 50.8±8.8%, respectively. However, commercial naked PEI/siRNA complexes at the same weight ratio (the ratio of nitrogen in PEI/phosphate in siRNA=1.4) as NP-PEIb-siRNA-CTX did not showed any gene silencing effect in 10% serum medium (data not shown). Previous study also indicated that naked PEI/siRNA complexes showed negligible gene silencing effect up to nitrogen in PEI/phosphate in siRNA ratio of 8 in the presence of 10% serum. NP-siRNA without PEI coating showed negligible gene silencing or cytotoxic effect, which is probably due to the limited cellular internalization and escape from endosomes after intracellular uptake (data not shown).

In the practice of this embodiment of the invention, the primary amines of PEI were blocked by citraconic anhydride, which showed excellent biocompatibility. The resultant PEIb exhibited no cytotoxic effect up to a polymer concentration of 120 μg/mL, compared to acetylated PEI which showed severe cytotoxicity at a polymer concentration of 40 μg/mL. Moreover, the blocking of primary amine groups using citraconic anhydride is reversible in acidic pH conditions, enabling triggered cytotoxicity only at acidic pH of the common tumor microenvironment.

The nanoparticles conjugated with blocked PEI showed significant increase in surface charge at pH 6.4 compared to that at pH 7.4, while there were negligible differences in surface charge of NP—NH₂ and NP—SH at these two different pH conditions. This may be attributed to both the removal of blocking groups in primary amine groups and protonation of secondary/tertiary amines in PEI. This increase in surface charge in acidic pH resulted in enhanced intracellular uptake of nanovectors in the tumor microenvironment. The presence of CTX on NP-PEIb-siRNA-CTX further enhanced the nanovector uptake by target cells.

Nanoparticles designed for in vivo applications are preferred to have a diameter less than 100 nm to facilitate their navigation through the body, retain a long blood circulation time, and enhance permeability through blood vessels in the tumor microenvironment. The size of the resulting nanovector (NP-PEIb-siRNA-CTX) at the physiological condition was about 63 nm, suggesting that these nanovectors could be accumulated in tumor tissues via the enhanced permeability and retention effect. The increased hydrodynamic size of NP-PEIb-siRNA-CTX at pH 6.2 could be attributed to stretching out of CTX conjugated via dodecaethyleneglycol as a spacer. CTX is a cationic peptide with three lysines and two arginines per every 36 amino acids. The removal of blocking groups at acidic pH could result in increased surface charge, which may induce more favorable exposure of cationic CTX outward from NP-PEIb-siRNA-CTX via electrostatic charge repulsion.

In nanovector of the invention (FIG. 9C), the NP-PEIb-siRNA-CTX is highly negatively charged at the physiological pH but increases its surface charge in the acidic tumor extracellular microenvironment via both deblocking of some primary amine groups and protonation of remnant amine groups in PEI. Both the increased surface charge at acidic pH and the presence of targeting ligand CTX on the nanovector would enhance cellular uptake of the nanovector. Once the nanovector is endocytosed, the secondary and tertiary amine groups in PEI on the nanovector would facilitate its endosomal escape via the proton sponge effect. Upon escape from the endosome, the siRNA is released from the nanovector by cleavage of the disulfide linkage in the reductive cytosolic condition and provides therapeutic effect through gene silencing. A representative nanovector of the invention, NP-PEIb-siRNA-CTX, exhibited significantly increased gene silencing effect at pH 6.2, compared to that at pH 7.4 (FIG. 14B). In the representative nanovector, PEI was conjugated to NP via cleavable disulfide linkage, which could be dissociated in the reductive cytosol. The PEI with recovered primary amine groups could be released from NP in the cytosol, which might facilitate destabilization of mitochondrial membranes and activation of apoptotic signals for cytotoxic effect. There was no cytotoxic effect on C6 cells by acidic pH itself up to pH 5.4 (data not shown). This is likely due to cancer cells having their own mechanism of survival in acidic extracellular conditions. At pH 6.4, NP-PEIb-siRNA-CTX elicited the most significant cytotoxicity, as compared to other nanoparticles, as a result of both the deblocking of the primary amine groups on PEIb and enhanced cellular internalization of the nanovector by CTX. NP-PEIb-siRNA-CTX elicited a slightly higher cytotoxic effect (about 20%) than NP-PEIb-siRNA at pH 7.4, which might be attributed to the partial deblocking of the primary amine groups of PEIb in the endosomal/lysosomal acidic environment after the cellular uptake of NP-PEIb-siRNA-CTX. This can be attributed to the enhanced intracellular uptake of NP-PEIb-siRNA-CTX due to the presence of CTX as compared to NP-PEIb-siRNA.

In this embodiment, the superparamagnetic iron oxide nanoparticles were modified with three different functional molecules for enhanced cytotoxicity and gene silencing in cancer cells. NP-PEIb-siRNA-CTX retained sufficient magnetism for MR imaging. CTX conjugation to nanoparticles induced significantly enhanced intracellular uptake of nanovectors by C6 glioma cells. Moreover, cytotoxicity and gene silencing effect by NP-PEIb-siRNA-CTX were only observed in acidic pH conditions. These results suggest that this nanovector system, with dual targeting specificity and dual therapeutic effect, could be safely applied as a potential therapeutic imaging agent for the targeted treatment of glioma as well as other cancers.

As used herein, the term “about” refers to values +/−5% of the recited value.

The following examples are provided for the purpose of illustrating, not limiting, the invention.

### EXAMPLES

### Example 1

### The Preparation, Characterization, and Properties of Representative Nanoparticles: NP-siRNA-CTX with Low Molecular Weight PEI

In this example, the preparation of representative nanoparticles of the invention are described: nanoparticles coated with polyethylene glycol-grafted chitosan and a low molecular weight polyethylenimine, with siRNA and chlorotoxin covalently coupled to the coating. The particles are schematically illustrated in FIG. 1A. Their preparation is illustrated schematically in FIG. 1B.

All reagents were purchased from Sigma Aldrich (St. Louis, Mo.) unless otherwise specified.

PEG-Grafted Chitosan (PEG-g-Chitosan) Synthesis.

Chitosan used for this synthesis was obtained by oxidative degradation of as-received high molecular weight chitosan (Mw=190 kDa, Sigma, St. Louis, Mo.) with sodium nitrite (NaNO₂). The degradation was carried out by reacting 100 mM aqueous NaNO₂ solution with a 2 wt % chitosan solution (pH=4.5, dilute acetic acid) for 24 h at room temperature. PEG-g-chitosan (PEGylated chitosan) was prepared by alkylation of depolymerized chitosan followed by Schiff base formation. Methoxy PEG (Mn=2000 g/mole, Sigma Co.) was first oxidized into PEG-aldehyde and then reacted with primary amines of chitosan in the presence of sodium cyanoborohydride. The chemical structure and purity of the polymer were confirmed by HPLC and ¹H-NMR.

Alternatively, oxidative degradation of chitosan was carried out by reacting aqueous/acidic chitosan solution with nitrous acid at room temperature. 1 to 3 wt % of commercially available chitosan (Mw=100 to 600 kDa) solution was prepared in an aqueous solution with a hydrogen ion concentration (pH) of 1 to 5. Aqueous nitrous acid solution (20 to 200 mM) was then added into the chitosan solution dropwise and the reaction mixture was stirred for 0.5 to 50 h. After completion of the reaction, solution was neutralized by addition of ammonia, sodium hydroxide, or an anion exchange resin. Lyophilized chitosan oligomer was washed with 50 to 90% ethanol and dried under vacuum.

Coating Polymer Characterization.

The degree of chitosan PEGylation was determined using a UV spectroscopy method. Briefly, a dried sample of PEGylated chitosan was weighed, and subsequently dissolved in 0.1 N HCl solution. The absorbance of solutions at a wavelength of 201 nm was then measured using UV spectroscopy and compared to a standard of pure chitosan to determine chitosan content. The following formula was then applied to determine the degree of pegylation: PEG weight in solution=Total sample weight−measured chitosan weight. Using this method we determined the molar ratio of chitosan:PEG to be 2.2.

Nanoparticle Synthesis.

Nanoparticles coated with PEG-g-chitosan were synthesized by first dissolving 3.0 g of PEG-g-chitosan, prepared as described above, in 50 ml deionized H₂O followed by addition of an iron chloride solution (4.6 g FeCl₂.H₂O and 9.1 g FeCl₃ dissolved in 50 ml of deoxygenated deionized H₂O). This mixture was then heated to 40° C. under mechanical stirring and nitrogen bubbling. One hundred mL of 7% NH₄OH was then added to the polymer and iron chloride mixture at a rate of 100 ml per hr. The resulting black precipitate was dialyzed for 2-3 days in H₂O to remove unreacted reagents.

1.2-kDa PEI was then modified with succinimidyl iodoacetate (SIA; Molecular Biosciences, Boulder, Colo.) at a 1.2:1 molar ratio in thiolation buffer (0.1M sodium bicarbonate, 5 mM EDTA, pH 8.0) through N-hydroxy succinimide ester chemistry. Nanoparticles coated with chitosan-g-PEG were modified with 2-iminothiolane (2IT; Molecular Biosciences, Boulder, Colo.) by adding 30 mg of 2IT to 3 ml of nanoparticles (1 mg of Fe/ml).

Reactions were shielded from light and preceded under gentle shaking for one hour. After the one-hour incubation, excess 2IT was removed by gel permeation chromatography using a PD-10 desalting column (GE Healthcare, Piscataway, N.J.) equilibrated with thiolation buffer. The modified PEI was added to the purified nanoparticle (216 mg PEI/g Fe₃O₄) and reacted in the dark for >1 hr under gentle shaking. The resulting nanoparticle/PEI complex was stored at 4° C. and allowed to react overnight before removing excess PEI using S-200 Sephacryl resin (GE Healthcare, Piscataway, N.J.) equilibrated with HEPES buffer (20 mM, pH 7.4). The formed iron oxide nanoparticles coated with chitosan-g-PEG and subsequently modified with PEI modified are referred herein as NP.

siRNA Preparation.

siRNA sequences designed to knockdown GFP expression and modified with thiol (a set without thiol modification were also acquired) and Dy547 were purchased from Dharmacon, Lafayette, Colo.: 5′-GCAAGCUGACCCUGAAGUUCUU-3′-antisense (SEQ ID NO: 1) and 5′-GAACUUCAGGGUCAGCUUGCUU-3′-sense (SEQ ID NO: 2). These sequences were acquired with protected-thiol modifications on the 5′ end of the sense strand and with Dy547 modification on the 5′ end of the antisense strand. siRNA sequences were received as single strands and were annealed to their complementary strand in annealing buffer (12 mM potassium chloride, 1.2 mM HEPES, 0.04 mM magnesium chloride, pH 7.5) by incubating at 95° C. for five minutes, then 37° C. for 1 hr, and then stored at −20° C.

Nanoparticle-siRNA Complex (NP-siRNA) Formation.

A suspension of nanoparticle (NP) prepared as described above in thiolation buffer was prepared at a concentration of 1 mg of Fe/ml. Amine groups on the surface of NP were then modified with SIA by addition of 200 μg of SIA dissolved in DMSO. The annealed siRNA with protected-thiol was deprotected by adding 57.3 mg/mL Tris(2-carboxyethyl) phosphine hydrochloride (TCEP.HCl; Molecular Biosciences, Boulder, Colo.) at a 1:1 volume ratio. Both reactions proceeded in the dark with gentle rocking for 1 hr. After 1 hr, both the SIA-modified NP(NP-SIA) and the deprotected siRNA were purified using Zeba™ Micro Spin Desalting Columns (Thermo Fisher Scientific, Waltham, Mass.) equilibrated with thiolation buffer supplemented with 150 mM NaCl. NP-SIA and deprotected siRNA were then mixed in PBS (5 mM EDTA, pH 7.4) at concentrations corresponding to the wt:wt (Fe mass of NP:siRNA mass) ratios tested (0:1, 0.1:1, 0.5:1, 1:1, 5:1, 10:1, 20:1) and immediately vortexed. The solutions were incubated overnight at 4° C. to allow the formation of NP-siRNA complexes.

Nanoparticle-siRNA-Chlorotoxin Complex (NP-siRNA-CTX) Formation.

A 1 mg/mL solution of chlorotoxin (CTX; Alamone Labs, Jerusalem, Israel) was prepared in thiolation buffer and reacted with 2IT at a 1.2:1 molar ratio of 2IT:CTX for 1 hour in the dark. NP-siRNA complexes were reacted with SM(PEG)₁₂ (Thermo Fisher Scientific, Waltham, Mass.) at 216 μg of SM(PEG)₁₂/mg Fe₂O₃ in the dark with gentle rocking for 30 minutes. NP-SM(PEG)₁₂ was then reacted with CTX-2IT at 1 μg CTX per 4.5 μg Fe for one hour in the dark. The resultant NP-siRNA-CTX was purified using Zeba™ Micro Spin Desalting Columns equilibrated with PBS, and stored at 4° C.

siRNA Binding Assay.

A 4% low melting point agarose gel was prepared with 0.05 μg/mL ethidium bromide. While maintaining a uniform concentration of siRNA, samples of NP:siRNA complexes were prepared at NP:siRNA weight ratios (Fe mass of NP:siRNA mass) ranging from 0:1 to 20:1. siRNA binding was analyzed by gel electrophoresis at 55 V for 90 min. Images were acquired on a Gel Doc XR (Bio-Rad Laboratories, Hercules, Calif.).

siRNA Release Assay.

NP-siRNA complexes were prepared at the optimal ratios determined from the binding assay (10:1, w/w), and reacted in 100 mM glutathione for 90 min at 37° C. Samples were then treated with heparin (1,000 units/ml, 10 μL heparin/μg siRNA) and incubated for 30 min at room temperature to block the electrostatic interaction between the NP and siRNA. siRNA release was analyzed on a 4% low melting point agarose gel containing 0.05 μg/mL ethidium bromide, running at 55 V for 90 min. Images were obtained on a Gel Doc XR. siRNA release was quantified using the Quantity One software package (Bio-Rad Laboratories, Hercules, Calif.).

CTX Binding Assay.

To quantify the degree of CTX attachment to nanoparticles, NP-siRNA-CTX were prepared as described above without purification of unbound CTX through S-200 sephacryl resin. Free, unreacted CTX was separated from the CTX conjugated to NPs through SDS-PAGE and quantified using the Quantity One software package and a standard curve of known concentrations of CTX. CTX conjugated to NPs was calculated by subtracting the amount of free CTX from the total amount of CTX in the reaction.

Nanoparticle Characterization.

Nanoparticle samples were prepared in 20 mM HEPES buffer (pH 7.4) to a concentration of 100 μg/mL, and then analyzed for hydrodynamic size and zeta potential using a DTS Zetasizer Nano (Malvern Instruments, Worcestershire, UK). For stability studies, nanoparticle samples were diluted to a concentration of 100 μg/mL in the indicated solution, then analyzed for hydrodynamic size using a DTS Zetasizer Nano, and imaged using a digital camera.

Cell Culture.

C6 rat glioma cells (ATCC, Manassas, Va.) were maintained in Dulbecco's Modified Eagle Medium (DMEM) (Invitrogen, Carlsbad, Calif.) supplemented with 10% FBS (Atlanta Biological, Lawrenceville, Ga.) and 1% antibiotic-antimycotic (Invitrogen, Carlsbad, Calif.) at 37° C. and 5% CO₂. Enhanced Green Fluorescent Protein (EGFP) fluorescing C6 cells (GFP+ C6) were produced by stably transfecting C6 cells with the pEGFP-N1 vector using the Effectene transfection reagent (Qiagen, Valencia, Calif.) following the manufacturer's protocol. 48 hrs post-transfection, cells were sorted using a FACS Vantage and maintained in fully supplemented DMEM with 1 mg/ml G-418.

Cell Transfection.

The day before transfection, cells were plated at 200,000 cells per well in 12-well plates. For transfection of cells with NP-siRNA and NP-siRNA-CTX formulations, cells were treated with 2 μg/mL of siRNA for 2 hrs under normal cell culture conditions. After the 2-hour incubation the media was replaced and cells were incubated for an additional 48 hrs before analysis. For transfection of cells with free siRNA, cells were treated with siRNA at 2 μg/ml for 48 hrs under normal cell culture conditions. For transfection of cells with PEI/siRNA, siRNA (20 μg/ml, serum free DMEM) and 25-kDa hyperbranched PEI (52 μg/ml, serum free DMEM) were mixed and allowed to form the PEI/siRNA complex for 15 min, and cells were then treated with the complexes (2 μg of siRNA/ml in serum free DMEM) for 24 hrs. After the 24-hour incubation cell culture media was replaced with serum contain DMEM, and cells were assayed after an additional 24 hours of growth. Transfections of cells with siRNA using Dharmafect 4 (Lafayette, Colo.) and Lipofectamine 2000 (Invitrogen, Carlsbad, Calif.) were performed according to the manufacturer's instructions.

Alamar Blue Cell Viability Analysis.

Cells were grown to confluence on 12-well plates and treated as described for cell transfection experiments. After treatment, samples were washed with PBS three times, and incubated for 1 hr with 10% Alamar blue (Invitrogen) in phenol-free DMEM (supplemented with 10% FBS and 1% antibiotic-antimycotic). The percent reduction of Alamar blue was determined following the manufacturer's protocol. Cell counts of treated and untreated samples were compared to determine percent viability of treated samples (untreated cells assumed to represent 100% viability).

Confocal Fluorescence Microscopy.

50,000 treated cells were plated on each of 24 mm glass cover slips and allowed to attach for 24 hrs. Cells were then washed with PBS and fixed in 4% formaldehyde (Polysciences Inc., Warrington, Pa.) for 30 min. Cells were then washed 3 times with PBS, and stained with membrane stain WGA-AF647 (Invitrogen, Carlsbad, Calif.) according to the manufacturer's instructions. Cover slips were then mounted on two microscope slides using Prolong Gold antifade solution (Invitrogen, Carlsbad, Calif.) containing DAPI for cell nuclei staining. Images were acquired on a LSM 510 Meta confocal fluorescence microscope (Carl Zeiss Inc., Peabody, Mass.) with the appropriate filters.

Endosomal Escape Assay.

C6 cells (200,000 cells/cover slip) were plated on 24 mm glass cover slips and allowed to attach for 24 hrs under normal cell culture conditions. Cells were then treated with the membrane impermeable dye Calcein (0.25 mM, Invitrogen), physical mixtures of calcein and NP-siRNA-CTX, or calcein and NP/siRNA. After 2 hrs, excess dye was washed off and samples were prepared for confocal fluorescence microscopy as described above. Fluorescence imaging was used to evaluate endosome escape (indicated by diffuse cytoplasmic calcein fluorescence).

Flow Cytometry.

Cells treated with a transfection reagent were washed with PBS, and detached using TrypLE Express (Invitrogen, Carlsbad, Calif.), and resuspended in PBS containing 2% FBS. At least 10,000 cells were then analyzed using a BD FACSCanto flow cytometer (Beckton Dickinson, Franklin Lakes, N.J.) and data analyses were performed using the FlowJo software package (Tree Star, Ashland, Oreg.).

Real-Time PCR.

For gene expression analysis, cells were removed from culture 48 hrs after transfection and RNA was extracted using the Qiagen RNeasy kit (Qiagen, Valencia, Calif.) following the manufacturer's protocol. mRNA reverse transcription (RT) was performed with a BioRad iScript cDNA synthesis kit (Bio-Rad, Hercules, Calif.). DNA transcripts were then probed using BioRad iQ SYBR Green Supermix with Qiagen QuantiTect Primer Assays for GFP and rat β-actin. Thermocycling was performed with a BioRad CFX96 Real-Time Detection System under the following conditions: 95° C. for 15 min, 45 cycles of denaturation (15 s, 94° C.), annealing (30 s, 55° C.), and extension (30 s, 72° C.). The relative expression of GFP was compared to the expression of β-actin and normalized to the untreated cells as a control.

In Vitro MRI.

Cell samples (1 million cells) were suspended in 50 μL of 1% agarose. For nanovector samples, 25 μL nanoparticles dispersed in PBS were mixed with 25 μL of 1% agarose. T₂ relaxation measurements were performed on a 4.7-T Bruker magnet (Bruker Medical Systems, Karlsruhe, Germany) equipped with Varian Inova spectrometer (Varian, Inc., Palo Alto, Calif.). A 5 cm volume coil and spin-echo imaging sequence were used to acquire T₂-weighted images. Images were acquired using a repetition time (TR) of 3000 ms and echo times (TE) of 13.6, 20.0, 40.0, 60.0, 90.0 and 120.0 ms. The spatial resolution parameters were acquisition matrix of 256×128, field-of-view of 35×35 mm, section thickness of 1 mm, and two averages. The T₂ map was generated by NIH ImageJ (Bethesda, Md.) based on the equation, SI=A*exp(−TE/T₂)+B, where SI is the signal intensity, TE is the echo time, A is the amplitude, and B is the offset. R₂ maps were generated by taking the reciprocal of T₂ maps.

### Example 2

### The Preparation, Characterization, and Properties of Representative Nanoparticles: NP-siRNA-CTX with Amine Blocked PEI

In this example, the preparation of representative nanoparticles of the invention are described: nanoparticles coated with polyethylene glycol-grafted chitosan and primary amine blocked polyethylenimine to which are covalently coupled siRNA and chlorotoxin. The particles are schematically illustrated in FIG. 1A. Their preparation is illustrated schematically in FIG. 9A.

Preparation and Characterization of Primary Amine Blocked PEI.

Two types of amine blocked PEIs (BPEI), the less amine group blocked PEI (PEIa) and highly amine blocked PEI (PEIb), were prepared for this study. After performing 1/10 dilution of citraconic anhydride with dimethyl sulfoxide, predetermined amount of citraconic anhydride (0.33 mg, 0.66 mg) were reacted with branched PEI (MW25k, 1 mg) in PBS for 2 hrs at room temperature for the preparation of PEIa and PEIb resulting in citraconic anhydride/primary amine group in PEI with molar ratios of 0.42 and 0.84, respectively. To inhibit intra- and intermolecular ionic interactions, reaction was performed in the presence of high salts (0.5 M NaCl). After citraconylation of PEI, the remaining amine groups were activated with SPDP (2.3 mg) overnight at room temperature to prepare pyridyldithiol modified BPEI. After the reaction, the solution was purified using Zeba™ Spin Desalting Columns (MWCO 7k) to remove excess SPDP. The relative amounts of primary amine groups in both BPEIs and pyridyldithiol activated BPEIs were determined by the fluorescamine assay according to the manufacturer's protocol. Ten microliters of fluorescamine solution in acetone at a final concentration of 7 mg/ml were mixed with each polymer solution in PBS (100 μL) and protected from light. After incubating for 10 min at room temperature, the fluorescent intensity was measured at excitation and emission wavelengths of 390 nm and 475 nm, respectively. To examine the cleavage of the blocking group from BPEI, pyridyldithiol modified PEIb was incubated in HEPES buffer at pH 7.4, 6.4, 4.5 and 0.3 for 24 hrs. The amount of exposed amine groups at each pH condition was determined by the fluorescamine assay (Oh, I. K.; Mok, H.; Park, T. G., Folate immobilized and PEGylated adenovirus for retargeting to tumor cells. Bioconjug. Chem. 2006, 17 (3), 721-727).

To examine cell cytotoxicity of both naked PEI and modified PE1, C6 cells were seeded on 24-well plates at a density of 1×10⁵ cells per well, and treated with each polymer at various concentrations (0, 1, 2, 4, 8, 16, 32, 64, 128 μg/ml) for 24 hrs in DMEM medium supplemented with 10% FBS. After incubation, cells were washed with PBS three times and treated with DMEM containing 10% Alamar Blue solution for 2 hrs, according to the manufacturer's protocol. Fluorescence intensity in each sample was measured by Spectra Max microplate reader with an excitation and emission wavelength at 470 nm and 486 nm, respectively. The relative cell viability was determined by assuming untreated cells having 100% viability.

Preparation and Characterization of Multifunctional Iron Oxide Nanoparticles.

Oleic acid coated iron oxide nanoparticles with a 12-nm core diameter were synthesized via thermal decomposition of iron oleate complex and coated by PEG with terminal amine groups for the preparation of amine-functionalized nanoparticles (NP—NH₂), as described above in Example 1. After synthesis of NP—NH₂, the concentration of Fe was analyzed by inductively coupled plasma atomic emission spectroscopy (ICP-AES). The number of amine groups per NP (˜70 NH₂/NP) was determined by quantifying pyridine-2-thione using SPDP, according to the manufacturer's protocol. The NP (280 μg) in 0.1 M sodium bicarbonate buffer (pH 8.5) was mixed with Traut's reagent (245.7 μg) for the preparation of thiol modified NP(NP—SH). Excess Traut's reagent was removed using Zeba™ Spin Desalting Columns (MWCO 40k) according to the manufacturer's protocol. Pyridyldithiol activated PEIb (75 μg) was added to NP—SH at a 1:50 molar ratio of NP:PEIb and reacted for 24 hrs to prepare amine-blocked PEI coated NP(NP-PEIb). To prepare siRNA with a free thiol group (siRNA-SH), 1000 μL of 1M dithiothreitol (DTT) in DW was added to 100 μL of 5′-end thiol-blocked siRNA (0.96 mM) in PBS. The final pH of the solution was adjusted to 8.0 using 5N NaOH. After an overnight reaction, the reactant was purified using Zeba™ Spin Desalting Columns (MWCO 7k). Both siRNA-SH (134.4 μg) and SM(PEG)₂ (4.25 μg) were added to NP-PEIb and reacted for 10 hrs for the preparation of NP-PEIb-siRNA at a 1:100 molar ratio of NP:siRNA.

Chlorotoxin (CTX, MW8000) was recombinantly synthesized in Escherichia coli (Deshane, J.; Garner, C. C.; Sontheimer, H., Chlorotoxin inhibits glioma cell invasion via matrix metalloproteinase-2. J Biol Chem 2003, 278 (6), 4135-44). Talon Resin pure CTX was further purified using size exclusion liquid chromatography and characterized using polyacrylamide gel electrophoresis. CTX (500 μg) in PBS was reacted with Traut's reagent at a 1:1 molar ratio of Traut's reagent:CTX for 1 hr at room temperature. The pH of the reactant solution was adjusted to 8.0 using 1N NaOH. Thiolated CTX (200 μg) and SM(PEG)₁₂ (12.97 μg) were added to NP-PEIb-siRNA at a 1:200 molar ratio of NP:CTX and reacted overnight. The resultant NP-PEIb-siRNA-CTX was purified using Zeba™ Spin Desalting Columns (MWCO 40k) equilibrated with PBS, and stored at 4° C. TEM samples were observed on a Phillips CM100 TEM (Philips, Eindhoven, The Netherlands) operating at 100 kV. The surface charge and hydrodynamic size of nanoparticles were analyzed using a Malvern Nano Series ZS particle size analyzer (Worcestershire, UK).

Quantification of Intracellular Iron Content.

C6 cells were maintained in DMEM (Invitrogen, Carlsbad, Calif.) supplemented with 10% FBS (Atlanta Biologicals, Lawrenceville, Ga.) and 1% antibiotic-antimycotic (Invitrogen, Carlsbad, Calif.) at 37° C. and 5% CO₂. C6 cells stably expressing GFP (GFP+ C6) were prepared by transfecting C6 cells with the pEGFP-N1 vector using Effectene transfection reagent (Qiagen, Valencia, Calif.) according to the manufacturer's protocol. The day before transfection, cells were plated at a density of 3×10⁵ cells per well in 12-well plates. Cells were then treated with five types of iron oxide nanoparticles, NP, NP—SH, NP-PEIb, NP-PEIb-siRNA, and NP-PEIb-siRNA-CTX, at an Fe concentration of 4 μg/mL for 6 hrs at pH 7.4 and pH 6.4. After incubation, cells were washed with PBS and lysed with 400 μL of 50 mM NaOH solution. Intracellular Fe content was determined by the colorimetric ferrozine-based assay (Riemer, J.; Hoepken, H. H.; Czerwinska, H.; Robinson, S. R.; Dringen, R., Colorimetric ferrozine-based assay for the quantitation of iron in cultured cells. Anal Biochem 2004, 331 (2), 370-5). Absorbance of each sample was measured at 562 nm using the Spectra Max microplate reader. The number of cells was also determined by the Coomassie Blue assay according to the manufacturer's protocol.

In Vitro MRI.

C6 cells (5×10⁵ cells/well) were seeded on a 12-well plate 24 hrs before treatment. Cells were treated with nanoparticles in growth media (20 μg Fe/mL) at pH 6.4 and incubated for 24 hrs. After incubation, cell pellets were prepared by centrifugation at 1500 g for 5 min. Cell pellets were resuspended in 50 μL of 1% agarose. For nanoparticle samples, 25 μL of nanoparticles at various Fe concentrations (0.125, 1.25, 2.5, 5, 10 μg/mL) in PBS were mixed with 25 μL of 1% agarose. T2 relaxation measurements were performed on a 4.7-T Bruker magnet (Bruker Medical Systems, Karlsruhe, Germany) equipped with Varian Inova spectrometer (Varian, Inc., Palo Alto, Calif.). A 5 cm volume coil and the spin-echo imaging sequence were used to acquire T2-weight images. Images were acquired using a repetition time (TR) of 3000 ms and echo times (TE) of 13.6, 20.0, 40.0, 60.0, 90.0 and 120.0 ms. The spatial resolution parameters were: acquisition matrix of 256×128, field-of-view of 35×35 mm, section thickness of 1 mm and two averages. The T2 map was generated by NIH ImageJ (Bethesda, Md.) based on the equation, SI=A exp(−TE/T2)+B, where SI is the signal intensity, TE is the echo time, A is the amplitude, and B is the offset. R2 maps were generated by taking the reciprocal of T2 maps.

Cell Viability and Gene Silencing Effect.

Cells were seeded on 24-well plates at a density of 1×10⁵ cells per well, and treated with nanoparticles (8 μg Fe/ml) for 48 hrs in DMEM supplemented with 10% FBS at pH 7.4 and pH 6.4. After incubation, cell viability was examined by the Alamar blue assay. To quantify the degree of GFP gene silencing, the cells were transfected with various amounts of nanoparticles (0, 0.5, 1, 2, 4, 8 μg Fe) for 24 hrs in the presence of serum at pH 7.4 and pH 6.4 and incubated for an additional 24 hrs after changing medium with DMEM with 10% FBS. After transfection, cells were washed with PBS three times and treated with a cell lysis solution (1% Triton X-100 in PBS). GFP protein expression was measured at an excitation and an emission wavelength of 488 and 520 nm, respectively. The extent of GFP fluorescence was normalized by the total viable cells, which was determined by the Alamar Blue assay. Relative GFP expression levels were then calculated based on the GFP expression percent of non-transfected C6 cells used as a 100% control.

While illustrative embodiments have been illustrated and described, it will be appreciated that various changes can be made therein without departing from the spirit and scope of the invention.

The embodiments of the invention in which an exclusive property or privilege is claimed are defined as follows:

1. A nanoparticle, comprising:
(a) a core having a surface and comprising a core material; and
(b) a coating on the surface of the core, the coating comprising
(i) a copolymer comprising a chitosan and a poly(ethylene oxide) oligomer; and
(ii) a polyethylenimine having an average molecular weight from about 500 to about 10,000 g/mole, or
(iii) a polyethylenimine having primary, secondary, and tertiary amine groups, wherein at least a portion of primary amine groups are modified to provide amide groups.

2. The nanoparticle of claim 1 further comprising a therapeutic agent covalently coupled to the coating.

3. The nanoparticle of claim 1 further comprising a targeting agent covalently coupled to the coating.

4. The nanoparticle of claim 1 further comprising a therapeutic agent covalently coupled to the coating and a targeting agent covalently coupled to the coating.

5. The nanoparticle of claim 1, wherein the polyethylenimine having an average molecular weight from about 500 to about 10,000 g/mole has an average molecular weight from about 500 to about 2,000 g/mole.

6. The nanoparticle of claim 1, wherein the polyethylenimine having at least a portion of primary amine groups modified to provide amide groups has an average molecular weight from about 600 to about 60,000 g/mole.

7. The nanoparticle of claim 1, wherein the portion of primary amine groups modified to provide amide groups introduce carboxylate groups to the polyethylenimine.

8. The nanoparticle of claim 1, wherein the polyethylenimine modified to provide amide groups is reactive under acidic conditions to reverse the modification and regenerate the primary amine groups.

9. The nanoparticle of claim 2, wherein the therapeutic agent is selected from the group consisting of a small organic molecule, a peptide, an aptamer, a protein, and a nucleic acid.

10. The nanoparticle of claim 2, wherein the therapeutic agent is an RNA or a DNA.

11. The nanoparticle of claim 2, wherein the therapeutic agent is an siRNA.

12. The nanoparticle of claim 2, wherein the therapeutic agent is covalently coupled to the coating is coupled through a cleavable linkage.

13. The nanoparticle of claim 3, wherein the targeting agent is selected from the group consisting of a small organic molecule, a peptide, an aptamer, a protein, and a nucleic acid.

14. The nanoparticle of claim 3, wherein the targeting agent is selected from the group consisting of a chlorotoxin, RGD, and VHPNKK.

15. The nanoparticle of claim 1 further comprising a fluorescent agent.

16. The nanoparticle of claim 1, wherein the core material is a magnetic material.

17. The nanoparticle of claim 1, wherein the copolymer is a graft copolymer having a chitosan backbone and poly(ethylene oxide) oligomer side chains.

18. A composition, comprising a nanoparticle of claim 4 and a carrier suitable for administration to a warm-blooded subject.

19. A method for detecting cells or tissues by magnetic resonance imaging, comprising:
(a) contacting cells or tissues of interest with a nanoparticle of claim 4; and
(b) measuring the level of binding of the nanoparticle, wherein an elevated level of binding, relative to normal cells or tissues, is indicative of binding to the cells or tissues of interest.

20. A method for treating a tissue, comprising contacting a tissue of interest with a nanoparticle of claim 4.

21. A method for silencing or reducing the expression level of a gene, comprising contacting a cell of interest with a nanoparticle of claim 4.


SEQUENCE LISTING (2 sequences)

SEQ ID NO: 1 - RNA, 22 nt, Artificial Sequence
  Synthetic
gcaagcugacccugaaguucuu

SEQ ID NO: 2 - RNA, 22 nt, Artificial Sequence
  Synthetic
gaacuucagggucagcuugcuu